(19) United States
(12) Patent Application Publication
(10) Pub. No.: US 2004/0001842 A1
(43) Pub. Date: Jan. 1, 2004

(54) Immunogenic compositions to the CCK-B/gastrin receptor and methods for the treatment of tumors

(76) Inventors: Dov Michaeli, Larkspur, CA (US); Martyn Caplin, London (GB); Susan A. Watson, Edwalton (GB); Stephen Grimes, Davis, CA (US)

Correspondence Address: WHITE & CASE LLP, PATENT DEPARTMENT, 1155 AVENUE OF THE AMERICAS, NEW YORK, NY, 10036, US

(21) Appl. No.: 10/323,692

(22) Filed: Dec. 19, 2002

Related U.S. Application Data
(63) Continuation-in-part of application No. 09/076,372, filed on May 12, 1998, now Pat. No. 6,548,066.
(60) Provisional application No. 60/046,201, filed on May 12, 1997.

(51) Int. Cl. A61K 39/00; C07K 14/705

(52) U.S. Cl. 424/185.1; 530/324; 530/326

(57) ABSTRACT

The invention concerns immunogens, immunogenic compositions and method for the treatment of gastrin-dependent tumors. The immunogens comprise a gastrin receptor immunomimic peptide conjugated to an immunogenic carrier. The immunogens are capable of inducing antibodies in vivo which bind to the gastrin-receptor (GR) in gastrin responsive malignant or premalignant tumor, thereby preventing growth stimulating peptide hormones from binding to the receptors, and inhibiting tumor cell growth. The invention also comprises specific antibodies against the gastrin-receptor for passive immunization. Furthermore, the invention comprises cytotoxic molecule derivatized anti-GR antibodies. The invention also concerns diagnostic methods for detecting gastrin-dependent tumors in vivo or from a tissue biopsy using the antibodies of the invention. Active and passive immunization can be combined providing an immune response against GR, G17 and/or G17-Gly.

### CROSS REFERENCE TO RELATED APPLICATION

This application is a continuation in-part of Ser. No. 09/076,372, which claims the benefit under 35 U.S.C. § 119(e), of U.S. Provisional Application No. 60/046,201 filed on May 12, 1997.

### BACKGROUND OF THE INVENTION

Gastrin is a peptide hormone which occurs in two forms, tetratriacontagastrin (G34) and heptadecagastrin (G17), and is synthesized and secreted by specialized cells, G cells, that are located in the stomach antrum. The hormone is secreted into the circulating blood and binds to specific cells in the stomach, namely, enterochromaffin-like (ECL) and parietal cells, that indirectly or directly affect stomach acid output. Historically, gastrin hormones have been associated with the stimulation of gastric acid secretion (Edkins, J. S. 1905). (The full citations for the references cited herein are provided in the Reference section preceding the claims.) In recent years, evidence has accumulated that gastrin may act as a trophic factor within the gastrointestinal tract (Johnson, L. 1997) and that it can promote the growth of gastrointestinal cancers (Watson et al. 1989, Dickinson, C. J. 1995), as well as non-gastrointestinal cancers including small cell carcinoma of the lung (Rehfeld et al. 1989). In the post-translational processing of gastrin, it is the “mature” carboxy-amidated form that binds to a cholecystokinin B/gastrin receptor with high affinity via its five carboxy-terminal amino acids (Kopin et al. 1992). The CCK-B/gastrin receptor (GR) is a trans-membrane protein which is coupled via a G protein to intracellular signal transduction pathways that in turn control the expression of various genes.

The CCK-B/gastrin receptor belongs to a family of G protein-coupled receptors with seven transmembrane domains with equal affinity for both CCK and gastrin (Soll et al. 1984). This receptor was named a CCK type-B receptor because it was found predominantly in the brain (Wank et al. 1992). The receptor was subsequently found to be identical to the peripheral CCK/gastrin receptor (GR) in the parietal and ECL cells of the stomach (Nakata et al. 1992). This receptor has been well characterized in a number of normal (Fourmy et al. 1984, Grider et al. 1990) and tumor tissues (Singh et al. 1990, Watson et al. 1993), and extensively studied using the rat pancreatic adenocarcinoma cell line AR42J (Scemama et al. 1987). The AR42J GR cDNA has been cloned and sequenced, and it is more than 90% homologous in DNA sequence to the GR in rat and human brain, and more than 84% homologous in sequence to the canine parietal cell GR cDNA (Wank, S. A. 1995), demonstrating a high sequence homology even between species.

It has been shown that several types of tumors, e.g., colorectal, stomach, pancreatic and hepatocellular adenocarcinomas possess GR in their plasma membranes and that they respond to gastrin with powerful cellular proliferation (Rehfeld, J. F. 1972, Upp et al. 1989 and Watson et al. 1993). More recently, it has been discovered that many of these cancer cells also secrete gastrin and thus effect an autonomous proliferative pathway (Van-Solinge et al. 1993, Nemeth et al. 1993, Seva et al. 1994 and 1995).

The peptide hormones Gastrin 17 (G17) and Gastrin 34 (G34) bind to the GR on the cell membrane of normal cells. However, it has been found that G17, and not G34, stimulates the growth of gastrin-dependent cancer cells. Serum-associated G17, in particular, has the potential to stimulate the growth of colorectal tumors in an endocrine manner mediated by CCK-B/gastrin receptors (Watson et al. 1993 and 1996) in the tumor cells. G17 appears to be particularly implicated in stimulating the growth of colorectal adenocarcinomas due to a possible increased affinity for the GR on the tumor cells, over other gastrin hormone species (Rehfeld 1972). The GR were found to be expressed in a high affinity form on 56.7% of human primary colorectal tumors (Upp et al. 1989). It has been postulated that a potential autocrine loop may also exist due to endogenous production of precursor gastrin peptides by such tumors (Van-Solinge et al. 1993 and Nemeth et al. 1993). The resulting G17 ligand/receptor complex stimulates cell growth by way of secondary messengers for regulating cell function (Ulrich et al. 1990). The binding of G17 to the GR leads to activation of phosphatidyl inositol breakdown, protein kinase C activation with a resultant increase in intracellular calcium ion concentration, as well as the induction of c-fos and c-jun genes via mitogen-activated protein kinase, which has been implicated in the regulation of cell proliferation (Todisco et al. 1995). Additionally, gastrin binding to the GR has been associated with the subsequent increase in phosphorylation by a tyrosine kinase, pp125FADK (focal adhesion kinase), which may also have a role in the transmission of mitogenic signals (Tanaguchi et al. 1994).

A number of high affinity CCK-B/gastrin receptor antagonists have been evaluated therapeutically both in vitro and in vivo in a number of experimental gastrointestinal cancers. For example, proglumide, a glutamic acid derivative (Seva et al. 1994; Harrison et al. 1990 and Watson et al. 1991a); Benzotript, an N-acyl derivative of tryptophan; L-365,260, a derivative of Aspercillin (Bock et al. 1989); and CI-988, a molecule that mimics the C-terminal pentapeptide sequence of CCK (Hughes et al. 1990) have been shown to effectively neutralize the effects of exogenous gastrin on gastrointestinal tumor growth both in vitro and in vivo (Watson et al. and Romani et al. 1994). However, these antagonists have severe toxic side effects and lack specificity as they block the action of all potential ligands of the receptor such as G34 and CCK in normal cells. Recently, highly potent and selective CCKB/gastrin receptor antagonists such as YM022 (Yuki et al., 1997) and YF476 (Takinami et al., 1997) have been also described.

Proglumide and Benzotript have been widely assessed in pre-clinical studies. The main problem with these compounds is their lack of potency, with relatively high concentrations required to displace G17 (Watson et al., 1992a; Watson et al., 1992b). Despite this, proglumide and benzotript inhibited the basal and gastrin-stimulated proliferation of a number of cell lines (Seva et al., 1990; Watson et al., 1991a). In addition, proglumide increased the survival of xenograft mice bearing the gastrin-sensitive mouse colon tumor, MC26, to 39 days in the treated animals from 25 days in the control animals.

Due to the low specificity of this class of gastrin antagonizing agents for the GR, the inhibition of tumor growth may not be effectively control with gastrin antagonists. Moreover, the cellular receptors which recognize and bind the gastrins do not bind all the inhibitors tested (Seva et al. 1994). Thus, if complete inhibition of gastrin binding to the receptor does not occur in the autocrine growth cascade, then the gastrin antagonists may be unable to block this mechanism of tumor growth promotion.

### SUMMARY OF THE INVENTION

A different approach to treating tumors bearing the GR is to induce the host's immune system to specifically attack the tumors by targeting the GR.

In this context, the present invention provides immunogenic compositions and immunological methods for the treatment of tumors that express receptors for gastrin. The method comprises the active or passive immunization of a patient with a CCK-B/gastrin receptor immunogen (GR-immunogen) or anti-CCK-B/gastrin receptor antibodies (anti-GR Ab). The antibodies induced by the immunogens are specific against the CCK-B/gastrin receptor (GR) on tumor cells and block the growth-promoting effects of gastrin on the receptors. The antibodies prevent the gastrin peptide hormones from binding to the GR on gastrin-dependent tumor cells; thus, the growth of the tumor is arrested. Moreover, the antibodies specific to the NH₂-terminal end of the receptor, upon binding to the receptor, are internalized and rapidly translocated into the cytoplasm and the nucleus of the tumor cells. This internalization can occur as early as 10 seconds after exposing the cells to the antibody and occurs independently of gastrin hormone binding. This rapid internalization of the antibody/receptor complex, in turn, causes the affected tumor cells to undergo apoptosis or suicide.

The immunogens of the invention comprise natural or synthetic peptides derived from the human GR, as the immunomimic portion of the immunogen. Although the immunization, passive or active, is directed primarily against extracellular domains GR diagnostic procedures on biopsy specimens can also utilize antibodies directed specifically against intracellular domains of the GR, for example so as to identify structural rearrangements of tumor expressed mutant sequences.

The invention thus provides a broad complement of GR-immunomimic peptide epitopes. The acronym for these gastrin receptor specific peptide epitopes is GRE (formerly designated as GRP) with numerical distinction between the various sequences, as described below.

The immunogens may also comprise a spacer peptide sequence attached to an end of the immunomimic peptide. The immunogen may also be conjugated to a protein carrier, such as diphtheria toxoid, tetanus toxoid, bovine serum albumin and the like.

In one embodiment of the invention, the method of immunization against the GR comprises active immunization, wherein a patient is immunized with an immunogen. The GRE-immunogen stimulates the production of antibodies against the GR on tumor cells. The antibodies produced by the GRE immunogens bind to the GR on tumor cells and effectively prevent the binding of the gastrin peptide hormones to the receptors, thereby inhibiting the autocrine growth-stimulatory pathway of tumor cell division and ultimately the growth of the tumor.

In addition, the active immunization, or also passive immunization can be administered in combination with chemotherapeutic treatment, using for example 5-FU/leucovorin.

In another embodiment of the invention, the method of treatment comprises passive immunization, in that exogenous antibodies against the GR are administered to a patient in a sufficient concentration to bind to the GR of the tumor cells, thereby blocking the binding of the ligands to the receptor. In another embodiment of this aspect of the invention, the antibodies for human therapy may be polyclonal or monoclonal antibodies which can be chimeric, humanized, or human antibodies which may be produced by methods well-known in the art. The anti-GR antibodies can be further purified by affinity chromatography using IgG-specific or GR-specific ligand-substitution matrices. Specific ligands are derived from GRE immunomimic peptides.

In addition, the anti-GR antibodies may be further conjugated to cytotoxic molecules such as cholera or diphtheria or ricin toxin, or to radioactive molecules labeled with a radionuclide, such as ⁹⁹Yttrium, ¹¹¹Indium, ¹²⁵Iodine and ¹³¹Iodine, to enhance the killing of the tumor cells. The anti-GR antibodies may also be attached to the surface of liposomes to target the liposomes to GR-positive tumors. Such targeted liposomes could contain anti-tumor agents including radionuclides and/or cytotoxic agents. In addition these GR-targeted liposomes could serve as vehicles of other agents directed against downstream targets of gastrin, such as e.g. COX-2 (cyclo-oxygenase-2) or HB-EGF (heparin binding epidermal growth factor-like growth factor).

The invention also provides a method for diagnosing a gastrin-responsive tumor, comprising the immunochemical detection of gastrin-responsive (gastrin receptor-containing) tumors from a tissue biopsy using the antibodies of the invention. The specific anti-GR antibodies of the invention can be labeled with a detection system utilizing compounds such as biotin, horseradish peroxidase and fluorescein to detect the gastrin receptors in the tumor tissue using standard immunochemical procedures.

The invention also provides a method for diagnosing a gastrin-dependent tumor, comprising the in vivo detection of gastrin-dependent (CCK-B/gastrin receptor-containing) tumors, using the anti-GR antibodies. The method comprises administering to a patient possessing a GR-expressive tumor an effective dose of radiolabeled anti-CCK-B/gastrin receptor antibodies via an intravenous injection, and imaging or detecting tumor cells having anti-GR antibodies bound to their cell membranes by standard scintigraphic scanning procedures. In this aspect of the invention, the anti-GR antibodies can be labeled with a detectable radionuclide such as ⁹⁹Technicium, ¹¹¹Indium, ⁹⁰Yttrium, and ¹³¹I.

### BRIEF DESCRIPTION OF THE DRAWINGS

FIGS. 1A and 1B illustrate schematic views of the CCK-B/gastrin receptor and its 7 transmembrane domains.

FIG. 2 shows data from ELISA assays with antibodies raised in rabbits immunized with an immunogen against GRE1 of the CCK-B/gastrin receptor.

FIG. 3 shows data from ELISA assays with antibodies raised in rabbits immunized with an immunogen against Peptide 4 of the CCK-B/gastrin receptor.

FIG. 4 is a graph showing data obtained from an inhibition ELISA used to assess the specificity of affinity-purified antibodies raised against GRE1-DT immunogen.

FIG. 5 is a bar graph showing data on the inhibition of the binding of ¹²⁵I-human G17 to AR42J cells by peptide inhibitors.

FIG. 6 is a bar graph of the cellular distribution of immunogold-labeled AR4-2J tumor cells.

FIG. 7 is a photograph of a Western blot analysis of protein extracts from nuclear membranes of adenocarcinoma cells using antibodies raised against GRE1.

FIG. 8 is a photograph of a Western blot analysis of protein extracts from extranuclear and plasma membranes of adenocarcinoma cells using antibodies raised against GRE1.

FIG. 9 is a plot graph illustrating the C170HM2 tumor weight of control and anti-CCK-B/gastrin receptor-treated animals.

FIG. 10 is a plot graph illustrating the cross-sectional area of C170HM2 tumors from control and anti-CCK-B/gastrin receptor-treated animals.

FIG. 11 is a bar graph showing the mean C170HM2 tumor weights of control and anti-CCK-B/gastrin receptor-treated animals.

FIG. 12 is a bar graph showing the mean cross-sectional area of C170HM2 tumors of control and anti-CCK-B/gastrin receptor-treated animals.

FIG. 13 is a bar graph showing the mean number of C170HM2 tumors in control and anti-CCK-B/gastrin receptor-treated animals.

FIG. 14 is a bar graph showing the median C170HM2 tumor weight of liver metastases, of control and anti-CCK-B/gastrin receptor-treated animals.

FIG. 15 is a bar graph showing the median cross-sectional area of C170HM2 tumors from control and anti-CCK-B/gastrin receptor-treated animals.

FIG. 16 is a bar graph showing the median C170HM2 tumor number in control and anti-CCK-B/gastrin receptor-treated animals.

FIG. 17 is a bar graph showing the mean and median liver C170HM2 tumor number in control and anti-CCK-B/gastrin-receptor-treated animals.

FIG. 18 is a bar graph showing the mean and median liver C170HM2 tumor weight in control and anti-CCK-B/gastrin-receptor-treated animals.

FIG. 19 is a bar graph showing the mean and median values for the cross-sectional area of C170HM2 liver tumor metastases in control and anti-CCK-B/gastrin-receptor antibody-treated animals.

FIG. 20 depicts a graph showing the concentration of radiolabeled ¹²⁵I-antibodies in C170HM2 liver tumor xenografts of control (normal rabbit serum) and anti-GRE1-treated nude mice.

FIG. 21 depicts a bar graph showing the mean C170HM2 liver tumor number per liver of xenografts of control and anti-GRE1-treated nude mice.

FIG. 22 depicts a bar graph showing the mean C170HM2 liver tumor weight of liver xenografts of control and anti-GRE1-treated nude mice.

FIG. 23 depicts Western blots of C170HM2 liver tumor xenograft proteins of control and anti-GRE1-treated nude mice.

FIG. 24 is a photograph of a histological section taken with a light microscope showing a hematoxylin/eosin-stained section of a C170HM2 liver xenograft of a control mouse.

FIG. 25 is a photograph of a histological section taken with a light microscope showing a hematoxylin/eosin stained section of a C170HM2 liver xenograft from a mouse treated with rabbit anti-GRE1 antibodies.

Previously GRP named peptide epitopes have been renamed GRE.

### DETAILED DESCRIPTION OF THE INVENTION

The methods of the invention are directed to the treatment of gastrin hormone-dependent tumors in animals, including humans, and comprise administering to a patient an anti-CCK-B/gastrin-receptor immunogen, which produces antibodies in the immunized patient that bind to the CCK-B/gastrin-receptor (GR) on the tumor cells, so as to prevent the binding of the hormone to the receptor in order to inhibit the growth-promoting effects of the hormone. The GR immunomimic peptides are advantageously selected to produce antibodies directed against externally accessible moieties or epitopes of the GR.

More importantly, from a clinical point of view, the immunogen is constructed to produce antibodies capable of forming the receptor/anti-GRE1 antibody complex which is rapidly internalized, traverses the cytoplasm and, enters the nucleus. This reaction apparently triggers the affected tumor cells to commit suicide (apoptosis).

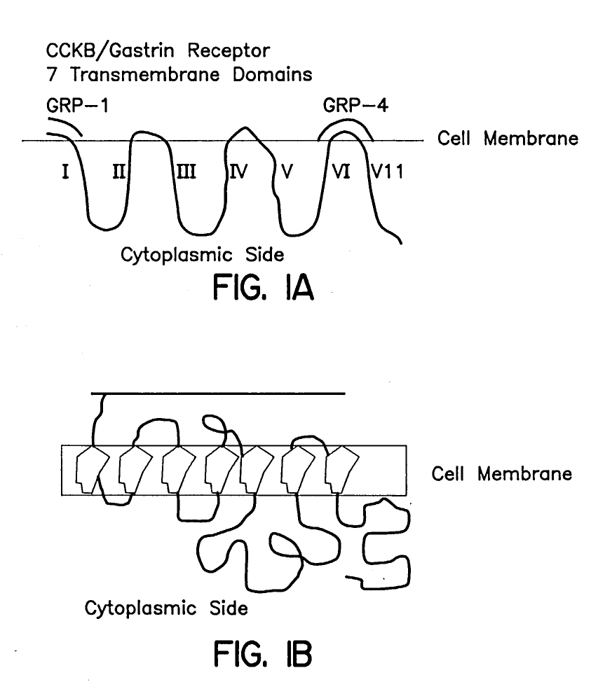

The immunogens comprise natural or synthetic peptides of the human GR which act as immunomimics. The variety of synthetic peptides that have been developed as the immunomimics is tabulated in Table I. These peptides, comprising effective epitopes, developed from the amino acid sequence of the GR, are capable of inducing antibodies that are cross-reactive with the GR of tumor cells both in vivo and in vitro. For example, the GRE1 epitope consists of amino acids 5 through 21 of the CCK-B/gastrin-receptor sequence: KLNRSVQGTGPGPGASL (SEQ ID NO.: 1 in the Sequence Listing). GRE1 is derived from the amino-terminal region of the receptor and is located on the extracellular surface of the cell membrane (see FIG. 1). Other sequences, such as GRE11, include the amino-terminus of the GR.

In another embodiment, the immunogen comprises epitope GRE4, which consists of the following amino acid sequence of the GR: GPGAHRALSGAPISF (SEQ ID NO.: 6 in the Sequence Listing); or GRE4-Ser (SEQ ID NO: 7) which is a synthetic spacer equipped peptide. GRE4 is part of the fourth extracellular domain of the receptor, and it, too, is on the extracellular surface of the plasma membrane (see FIG. 1).

In another embodiment, the immunogen comprises epitopes GRE9 or GRE10, which consists of amino acid sequences for a variant of the gastric receptor (GR) that is expressed almost exclusively by tumor cells. Antisera, purified IgG or monoclonal antibodies induced against this region, for example, can be used for diagnostic purposes on biopsy specimens collected from patients.

As listed in Table 1, a variety of synthetic peptides comprising different GR epitopes can induce anti-GR antibodies capable of blocking gastrin binding and receptor internalization potentially leading to apoptosis.

TABLE 1

Gastrin Receptor Immunomimic Peptides

| | | | |
|---|---|---|---|
| Epitope | Peptide Designation | AA | SEQ ID |

| | | | |
|---|---|---|---|
| GRE 1 | GRE 1-Ser | KLNRSVQGTGPGPGASLSSPPPPC | 4 |
| GRE 1 | GRE 1 EPT | KLNRSVQGTGPGPGASL | 1 |
| GRE 1 | GRE 1-Ala | KLNRSVQGTAPGPGASLAAC | 2 |
| GRE 1 | GRE 1- Gly 1 | CGGKLNRSVQGTGPGPGASL | 5 |
| GRE 4 | GRE 4-Ser | GPGAHRALSGAPISFSSPPPPC | 7 |
| GRE 4 | GRE 4 EPT | GPGAHRALSGAPISF | 6 |
| GRE 6 | GRE 6 | MELLKLNRSVQGC | 8 |
| GRE 9 | GRE 9-SSC | RDQDLGEADVWRASSC | 9 |
| GRE 10 | GRE 10-SSC | WERRSGGNWAGDWGDSPFSSC | 10 |
| GRE 11 | GRE 11 (GR1-22) | MELLKLNRSVQGTGPGPGASLC | 11 |
| GRE 11S | GRE 11 Ser | MELLKLNRSVQGTGPGPGASLSSPPPPC | 12 |
| GRE 12 | GRE 12 (GR 2-22) | ELLKLNRSVQGTAPGPGASLC | 13 |
| GRE 13 | GRE 13 (GR 3-22) | LLKLNRSVQGTGPGPGASLC | 14 |
| GRE 14 | GRE 14 (GR 4-22) | LKLNRSVQGTGPGPGASLC | 15 |
| GRE 15 | GRE 15 (GR 5-22) | KLNRSVQGTGPGPGASLC | 16 |
| GRE 16 | GRE 16 ([GR 2-12]-SSC) | ELLKLNRSVQGSSC | 17 |
| GRE 17 | GRE 17 (GR 11-22) | GTGPGPGASLC | 18 |

For example, this spacer sequence is combined with the GRE1 epitope to comprise peptide GRE1 Ser (SEQ ID NO: 4) in Table 1.

The synthetic peptides GRE11 through GRE-15 include sequences from the N-terminus of GR starting with residue 1 (GRE11 (i.e. GR 1-22)), residue 2 (GRE12), residue 3 (GRE13), residue 4 (GRE14), and residue 5 (GRE15) all ending the sequence at residue 22. GRE16 is shorter peptide starting at residue 2 and ending at residue 12 plus carrying a carboxy terminal SSC spacer. GRE17 is a fragment of GRE1 starting with residue 11 and ending with residue 22. The GRE11S refers to the Ser-spacer extended form of GRE11.

The immunogens may also comprise an extension or spacer peptide suitable for projecting the immunomimic peptide away from the protein carrier and enhancing its capacity to bind the lymphocyte receptors. A suitable spacer peptide comprises the amino acid sequence SSPPPPC (Serine (Ser) spacer, SEQ ID NO.: 3 in the Sequence Listing). However, as for example shown in Table I, other spacer peptides would also be suitable. The spacer peptides are not immunologically related to the GR derived peptides and therefore are used to enhance, but not determine, the specific immunogenicity of the receptor-derived peptides.

As shown in Table 1, the various peptide immunogens can be optionally modified to carry a spacer peptide. In an effective immunogenic construct, synthetic peptide GRE1 can be modified to carry the spacer peptide at its amino terminus or its carboxy terminus. According to the inventive embodiments, for example, the modifications include, but are not limited to C-terminal SSPPPPC (Ser-Spacer) or AAC; or N-terminal CGG.

The immunomimic peptides, with or without the spacer, are conjugated to a protein carrier, such as diphtheria toxoid, via a cysteine residue at the carboxy terminal end.

The presence and density of GR on tumor cells in a patient can be determined by reacting labeled anti-receptor antibodies with a sample obtained from tumor biopsy. The anti-receptor antibodies can be obtained by immunizing an animal with the immunogen of this invention. The anti-receptor antibodies are labeled with either a radioactive tracer, a dye or a fluorescent label. In addition, the responsiveness of the tumor cells to gastrin can be evaluated in vitro from a tumor biopsy sample of the patient using standard techniques. Patients having tumors positive for the anti-GR antibody tag are typical candidates for treatment according to the methods of the invention.

An effective dosage ranging from 0.001 to 2 mg of the immunogenic composition is administered to the patient for the treatment of the gastrointestinal cancer. The effective dosage of the immunogenic composition should be capable of eliciting an immune response in a patient consisting of effective levels of antibody against the GR 1-3 months after immunization. Following the immunization of a patient, the effectiveness of the immunogens is monitored by standard clinical procedures, such as ultrasound and magnetic resonance imaging (MRI), to detect the presence and size of a tumor. The antibody titer levels against the receptor may also be monitored from a sample of blood taken from the patient. Booster immunizations should be given as required to maintain an effective antibody titer. Such treatment of gastrin-dependent cancers, such as stomach, liver, pancreatic and colorectal adenocarcinomas, according to this method, should result in inhibition of tumor growth and a decrease in size of the tumor.

The antibodies raised by the GR-peptide immunogens of the present invention may have anti-trophic effects against gastrin-dependent tumors by three potential mechanisms: (i) inhibition of gastrin binding to its receptor; (ii) degradation or disruption of the signal transduction pathway of tumor cell proliferation; (iii) induction of apoptosis (or cell suicide) in cells where receptor/antibody complexes are internalized and migrate into the nucleus; and immune response associated killing mechanisms, such as antibody dependent cellular cytotoxity or complement mediated lysis or epsonization.

In another embodiment of the invention, anti-GR antibodies are directly administered to a patient possessing a gastrin-responsive tumor. The exogenous antibodies specifically bind to the GR complement of the tumor cells. The binding of the antibodies to the receptors prevents the binding of gastrin to its receptor in the membranes of cells and, therefore, the growth signal for the gastrin-dependent tumor cells is inhibited and the growth of the tumor is arrested.

These exogenously produced antibodies may also be useful for killing tumor cells that bear the GR on their plasma membranes by delivering a toxic substance to the tumor cell. For example, suitable anti-CCK-B/gastrin antibodies for therapy are those reactive with extracellular domains 1 and 4 of the receptor protein shown in FIG. 1 as GRE1 and GRE4, respectively. Antibodies raised against GR epitopes, such as GRE11, GRE6, GRE9, GRE12, GRE13 or GRE14 specifically recognize and bind amino acid sequences of the receptor protein. The antibodies may be polyclonal, humanized, monoclonal or human monoclonal antibodies. The inhibition of tumor growth in this method of immunization is also monitored by ultrasound imaging and MRI and repeated immunizations are administered as required by the patient.

The antibodies can also be reactive fragments of such antibodies (i.e. F(ab)₂ or Fab¹)thereof, which effectively bind to the target receptor and may be produced by standard techniques such as those disclosed in U.S. Pat. Nos. 5,023,077; 5,468,494; 5,688,506; and 5,662,702, the disclosures of which are hereby incorporated by reference. The fragments may be produced by enzymatic digestion with papain or pepsin as known in the art. Alternatively, specific antigen binding fragments may be produced by recombinant DNA or solid state peptide synthesis.

The effectiveness of the antibodies inhibiting tumor cell growth and killing of tumor cells can be enhanced by conjugation to cytotoxic molecules. The cytotoxic molecules can be toxins, for example, cholera toxin, ricin, α-amanitin, or radioactive molecules labeled, for example with ¹²⁵I or ¹³¹I, or chemotherapeutic agents, as for example, cytosine arabinoside or 5-fluorouridine.

The anti-GRE antibodies, which may be affinity-purified, humanized or human, polyclonal or monoclonal, when conjugated to cytotoxic molecules, can therefore act as specific targeted carrier protein. The antibodies are understood to be used as purified IgG fractions or in further modified form, such as F(ab)₂ or Fab¹ fragments. Binding of the antibodies is therefore independent of the F_{c} fragment. The antigen binding moieties may also be capable of improved permeation of tumor tissue, or even, if needed, penetration of the human blood-brain barrier.

The anti-GRE antibodies can further be used as carriers of adjunctive elements of anti-cancer efficacy, including, but not limited to, taxane, cisplatin, oxiplatin, camptothecin/camptosar, rubetecan, cyclophosphamide, doxirubicin, mitomycin C, vincristin, vinblastine, etoposide, noscapine, carboplatin, 5-fluorouridine and further, gemcitabine or irinatecan.

Alternatively, the anti-GR antibodies can be incorporated in the liposomal membranes so as to target and transport substantial amounts of anti-cancer agents to the appropriate GR containing tumor cells. The liposomes are prepared by standard methods (see U.S. Pat. No. 4,691,006).

The various types and quantities of conjugated anti-GRE IgG carriers are selected for treatment, on the basis of need and anti-tumor efficacy, by the attending physician. In general, the unit dosages range from 0.020 mg to 500 mg protein, which range can be exceeded in the course of treatment in terms of frequency of administration.

In addition to antibodies radiolabeled with ¹²⁵I and ¹³¹I, the anti-GR antibodies can also be labeled with radionuclides such as ⁹⁹Technicium, ¹¹¹Indium and ⁹⁰Yttrium. In this aspect of the invention the antibodies are useful for the detection and diagnosing of tumors possessing GR in vivo, by administering these antibodies to the patient, and detecting bound antibodies on GR-containing tumor cells. After allowing the radio-labeled anti-GR antibodies to reach the tumor, about 1-2 hours after injection, the radioactive “hot spots” are imaged using standard scintigraphic procedures as previously disclosed (Harrison's Principles of Internal Medicine, Isselbacher et al. eds. 13ₜₕ Ed. 1994).

The compositions in which the immunogens are administered for the treatment of gastrin-dependent tumors in patients may be in a variety of forms. These include, for example, solid, semi-solid and liquid dosage forms, such as powders, liquid solutions, suspensions, suppositories, and injectable and infusible solutions. The suitable form depends on the intended mode of administration and therapeutic applications. The compositions comprise the present immunogens and suitable pharmaceutically acceptable components, and may include other medicinal agents, carriers, adjuvants, excipients, etc. Suitable adjuvants may include nor-muramyl dipeptide (nor-MDP, Peninsula Labs., CA), and oils such as Montanide ISA 703 (Seppic, Inc., Paris, France), which can be mixed using standard procedures. The compositions are advantageously in the form of unit dose. The amount of active compound administered for immunization or as a medicament at one time, or over a period of time, will depend on the subject being treated, the manner and form of administration, and the judgment of the treating physician.

According to the invention, the anti-GR antibodies of the invention for passive immunization are administered to a patient intravenously using a pharmaceutically acceptable carrier, such as a sterile saline solution, for example, phosphate-buffered saline.

Another embodiment of the invention provides a combination of treatment to inhibit the binding of gastrin or activation of gastrin-responsive cells. In particular, such an embodiment can provide immunization with a gastrin immunogen (U.S. Pat. No. 5,468,494) and simultaneous immunization with a gastrin receptor immunogen. Alternatively, the method can combine immunizations with anti-gastrin antibodies such as anti-G17 antibodies as well as anti-GR antibodies. These antibodies can be monoclonal that may be human or humanized animal antibodies. Furthermore, the antibodies can be modified with cytotoxic substances, as described. Active and passive immunizations can be advantageously combined such that the passive immunization would serve as an instant effective activity which can be eased out when the antibody titer due to active immunization is sufficiently high and effective.

The temporary relatively short term use of passive immunization can help avoid or reduce an anti-antibody immune rejection over time.

Therefore, the protocol could provide initial administration of anti-G17 and anti-GR antibodies combined with active immunization using G17-immunogen and for GR-immunogen.

The combination of inhibiting gastrin, such as, e.g. G17 or G17-Gly (glycine extended-G17) as well as the GR moieties will synergistically prevent activation of gastrin promoted other growth factors, as well as prevent enhanced expression of the GR in response to the redution of gastrin signal in the immunized patient or host.

As described below, active immunization with rat GRE1 epitope in combination with 5-fluorouridine (5FU) plus leucovorin enhanced necrosis of liver metastases of the gastrin receptor expressing rat tumor DHDK 12 in rats.

### EXAMPLE 1

Preparation of GRE1-DT and GRE4-DT Conjugates

CCK-B/gastrin-receptor peptides selected to provide immunomimic epitopes were prepared by standard solid state peptide synthesis. To make immunogens more capable of inducing specific immune responses each of GRE1 and GRE4 was synthesized containing the spacer sequence SSPPPPC (SEQ ID NO.: 3 in the Sequence Listing) at its carboxy terminus. These peptides were subsequently conjugated to amino groups present on the carrier, Diphtheria toxoid (“DT”), via the terminal peptide amino acid residue cysteine of the spacer utilizing a heterobifunctional linking agent containing a succinimidyl ester at one end and maleimide at the other end of the linking agent by either of Method A, Method B or Method C as described below.

Method A: As previously described in U.S. Pat. No. 5,023,077, the linking of Peptide 1 or 4 above and the carrier is accomplished as follows. Dry peptide was dissolved in 0.1 M Sodium Phosphate Buffer, pH 8.0, with a thirty-fold molar excess of dithiothreitol (“DTT”). The solution was stirred under a water saturated nitrogen gas atmosphere for four hours. The peptide containing reduced cysteine was separated from the other components by chromatography over a G10 Sephadex column equilibrated with 0.2 M acetic acid. The peptide was lyophilized and stored under vacuum until used. The carrier was activated by treatment with the heterobifunctional linking agent e.g. Epsilon-maleimidocaproic acid N-hydroxysuccinimide ester, (“EMCS”), in proportions sufficient to achieve activation of approximately 25 free amino groups per 10⁵ molecular weight of carrier. In the specific instance of diphtheria toxoid, this amounted to the addition of 6.18 mg of EMCS (purity 75%) to each 20 mg of diphtheria toxoid.

Activation of diphtheria toxoid was accomplished by dissolving each 20 mg aliquot of diphtheria toxoid in 1 ml of 0.2 M Sodium Phosphate Buffer, pH 6.45. Aliquots of 6.18 mg EMCS were dissolved into 0.2 ml of Dimethyl Formamide (“DMF”). Under darkened conditions, the EMCS was added dropwise in 50 microliter (“μl”) amounts to the DT with stirring. After 2 hours of incubation in darkness, the mixture was chromatographed on a G50 Sephadex column equilibrated with 0.1 M Sodium Citrate buffer, pH 6.0, containing 0.1 mM EDTA.

Fractions containing the EMCS activated diphtheria toxoid were concentrated over a PM 10 ultrafiltration membrane under conditions of darkness. The protein content of the concentrate was determined by either the Lowry or Bradford methods. The EMCS content of the carrier was determined by incubation of the activated carrier with cysteine-HCl followed by reaction with 10 mM of Ellman's Reagent 5,5′ dithio-bis (2-nitrobenzoic acid) 10 mM. The optical density difference between a blank tube containing cysteine-HCl and the sample tube containing cysteine-HCl and carrier was translated into EMCS group content by using the molar extinction coefficient of 13.6×10³ for 5-thio-2-nitrobenzoic acid at 412 nm.

The reduced cysteine content (—SH) of the peptide was also determined utilizing. Ellman's Reagent. Approximately 1 mg of peptide was dissolved in 1 ml of nitrogen gas saturated water and a 0.1 ml aliquot of this solution was reacted with Ellman's Reagent. Utilizing the molar extinction coefficient of 5-thio-2-nitrobenzoic acid (13.6×10³, the free cysteine —SH was calculated. An amount of peptide containing sufficient free —SH to react with each of 25 EMCS activated amino groups on the carrier was dissolved in 0.1 M Sodium Citrate Buffer, pH 6.0, containing 0.1 mM EDTA, and added dropwise to the EMCS activated carrier under darkened conditions. After all the peptide solution had been added to the carrier, the mixture was incubated overnight in the dark under a water-saturated nitrogen gas atmosphere.

The conjugate of the peptide linked to the carrier via EMCS was separated from other components of the mixture by chromatography over a G50 Sephadex column equilibrated with 0.2 M Ammonium Bicarbonate. The conjugate eluted in the column void volume was lyophilized and stored desiccated at 20° C. until used.

The resulting conjugate may be characterized as to peptide content by a number of methods known to those skilled in the art including weight gain, amino acid analysis, etc. Conjugates constructed of GRE1 and GRE4 with spacer and diphtheria toxoid produced by this method were determined to have an effective peptide/carrier ratio of 5-35 moles of peptide per 100 KD MW of carrier and all were considered suitable as immunogens for immunization of test animals. Usually, the range of the peptide from 10-30 moles per 100 KD MW of DT produced an effective immune response.

Method B: In a preferred method, conjugates comprising GRE1, GRE4 peptide or any other suitable peptide immunomimic of the GR coupled to DT, were prepared at room temperature as follows. Purified DT (400 mg) was dissolved in 20 ml of 0.5 M phosphate buffer, pH=6.6, saturated with nitrogen gas to give a DT solution of 20 mg/ml. The DT solution was placed in a 60 ml dark amber glass bottle (serving as a reaction vessel and filtration reservoir). EMCS coupling reagent (123.6 mg) was dissolved in 2.0 ml of dimethylformamide. The EMCS solution was added dropwise to the DT solution over a 15 minute period with continuous stirring. The bottle was capped, and the mixture was stirred at room temperature for an additional 1 hour 45 minutes, to form activated DT (M-DT). The M-DT was then purified by diafiltration using an Amicon Model TFC10 Thin-Channel Ultrafiltration System per operating manual I-113G with a XM50 diaflow ultrafiltration membrane. The M-DT was washed twice against volumes of 420 ml phosphate buffer, concentrating to 20 ml each time, then washed once against 420 ml of 0.1 M sodium citrate buffer, pH=6.0, containing 0.1 M EDTA, and concentrating the solution down to 20 ml.

For example, to make GRE1-DT conjugate, 2.02 ml of M-DT solution (containing 22.3 mg M-DT) was placed in a 10 ml dark amber glass vial, then 13 mg of GRE1 peptide was dissolved in the citrate buffer to give 40 mg/ml peptide and added dropwise to the M-DT solution with stirring. Alternatively, to make GRE4-DT conjugate, 2.21 ml of M-DT solution (containing 24.4 mg M-DT) was placed in a 10 ml dark amber glass vial, then 13 mg of GRE4 peptide was dissolved in the citrate buffer to give 40 mg/ml peptide and added dropwise to the M-DT solution with stirring.

The reactions were allowed to proceed overnight in the dark. Each conjugate was removed from the reaction vessels and separately dialyzed in 12,000-14,000 MW cutoff dialysis tubing against 5 changes of 500 ml of 0.1 M ammonium bicarbonate solution. Each conjugate was lyophilized. The conjugates were then analyzed by amino acid analysis and their peptide to DT substitution ratios were determined to be 21.8 peptides per 10⁵ MW of DT for GRE1-DT and 21.1 peptides per 10⁵ MW of DT for GRE4-DT.

Conjugates of GRE1 and 4 with spacer and DT produced by this method have been selected for an effective peptide/carrier ratio of 5-35 moles of peptide per 100 KD MW of carrier and are all considered suitable as immunogens. An effective ratio for producing an effective immune response ranges from 10-25 moles of peptide per 100 KD MW of DT. These methods may also be supplanted by the closed system conjugation as described in coassigned U.S. Pat. No. 6,359,114.

Furthermore, these method examples apply as well to other receptor peptides, many of which are disclosed herein.

Method C: This procedure refers to a closed system for continuous conjugation and purification of immunogens (or other derivatizations of proteins, such cytotoxic IgG). The system is described in coassigned U.S. Pat. No. 6,359,114, which disclosure is incorporated herein by reference in its entirety.

Preparation of Immunogenic Compositions

The present immunogens containing either GRE1 or GRE4 with or without spacer conjugated to DT were used to immunize rabbits. Immunogens were prepared as follows: Conjugate was dissolved in 0.15 M Sodium phosphate buffered saline, pH 7.3 to a concentration of 3.79 mg/ml. The conjugate solution was added to Montanide ISA 703 Adjuvant (Seppic, Inc.) in a 30:70 (wt:wt) ratio of conjugate solution to Montanide ISA 703, then the mixture was homogenized using a Silverson Homogenizer for 3 minutes at 8,000 RPM to form an emulsion containing 1 mg/ml of conjugate.

Immunization and Sample Collection

Rabbits were injected intramuscularly with 0.1 ml of immunogen consisting of 0.1 mg of either GRE1-DT, or GRE4-DT conjugate. Each rabbit was given injections of immunogen at 0 and 4 weeks. Blood was collected from each rabbit at 6 and 8 weeks of the experiment. Serum was prepared from each blood sample and stored at −20° C. until utilized in assays to determine the presence of anti-GR antibodies.

Enzyme-Linked Immunosorbent Assay (ELISA)

A solid-phase ELISA was used to screen for reaction or cross-reaction of antisera raised against Peptide 1 and Peptide 4 of each immunized rabbit. The ELISA was carried out by coating polystyrene 96 well plates (IMMULON II, Dynatech) with 25 μl/well of 10 μg/ml of Peptide 1 linked to bovine serum albumin (BSA) (“GRE1-BSA”), or Peptide 4 linked to BSA (“GRE4-BSA”) antigen in 0.1 M Glycine-HCl, pH 9.5 buffer. The plates were incubated overnight at 4° C., and subsequently washed in buffer.

Antisera obtained from the immunized rabbits were serially diluted to a range of 10⁻¹ to 10⁻⁸ in 1% BSA-FTA hemagglutination buffer, pH 7.2. Twenty five μl of test antiserum per well was incubated with each test peptide for 1 hr at room temperature. After incubation, the plates were washed thoroughly with buffer to remove any unbound antibody. Each well was treated with 25 μl of biotinylated goat anti-rabbit IgG (H+L) diluted 1:1000 in 1% BSA-FTA dilution buffer for 1 hour at room temperature. After washing the plates to remove unbound anti-rabbit reagent, each well was incubated for 1 hour at room temperature with 25 μl of avidin-alkaline phosphatase conjugate diluted 1:1000 in 1% BSA-FTA buffer. The plates were washed thoroughly to remove unbound avidin-alkaline phosphatase reagent, and incubated with 25 μl of 1 mg/ml of p-nitrophenylphosphate (“PNPP”) in 10% diethanolamine buffer containing 0.01% MgCl₂.6H₂O, pH 9.8. The plates were allowed to develop until the absorbance of the reaction at 490 nm wavelength reached an optical density between 0.8 to 1.5. To test the specificity of the antisera produced by the rabbits, rabbits were also immunized with DT and for ELISA testing, plates were coated with DT as antigen to determine the reactivity of the antisera produced against the carrier.

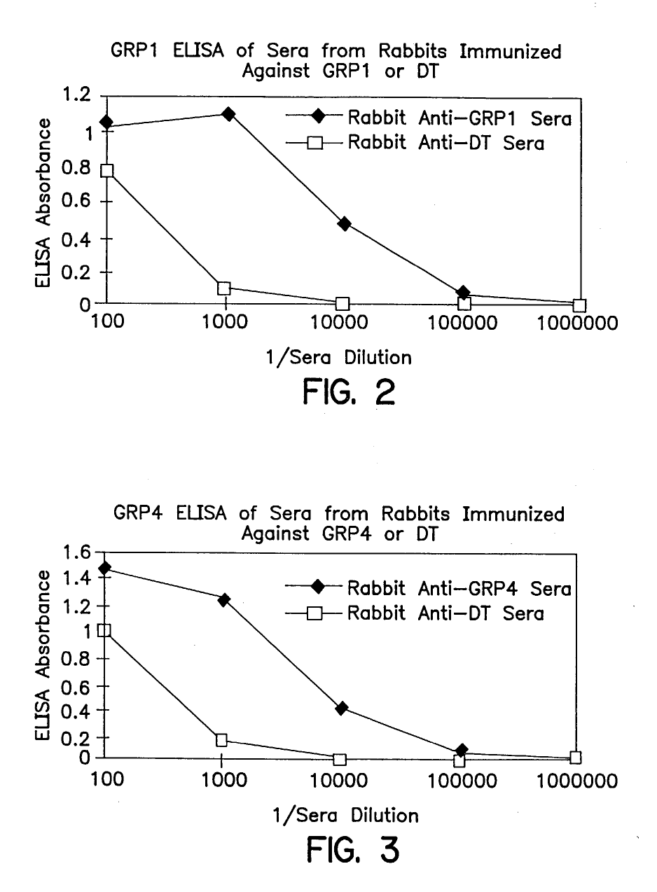

FIG. 2 shows the ELISA results using GRE1 and FIG. 3 shows the ELISA results using Peptide 4/GRE4 as the antigen. As seen in FIG. 2, the ELISA results show that the rabbits immunized with Peptide 1-spacer-DT conjugate produced high antibody titers which specifically bind to Peptide 1, as indicated by the antibody binding GRE1 even at high (1:100,000) dilutions of the antiserum. Similarly, FIG. 3 shows that rabbits immunized with Peptide 4-spacer-DT conjugate produced high titers of anti-GRE4 antibodies. As seen in FIGS. 2 and 3, the rabbits immunized against each peptide produced antibodies which bound specifically to each peptide at low antisera concentrations. The data indicate that the anti-GRE1 and anti-GRE4 antibodies have a large capacity for binding ligand GRE1 and GRE4 of the CCK-B/gastrin-receptor. The data also shows that immunization of rabbits with the present conjugates elicits powerful immune responses against GRE1 and GRE4, respectively. In addition, rabbits immunized with either GRE-1 or GRE-4 conjugate appeared and behaved normally and did not exhibit any symptoms of disease or pathologies during the experiments.

### EXAMPLE 2

The following experiments were performed to establish the specificity of antibodies raised in rabbits against the GRE1-DT peptide containing Ser spacer described in Example 1 using Method B. A series of tests were conducted to assess the specificity of rabbit antibodies induced by immunization with the GRE1-DT and affinity purified by immunoadsorption over a GRE1-Ser Sepharose column.

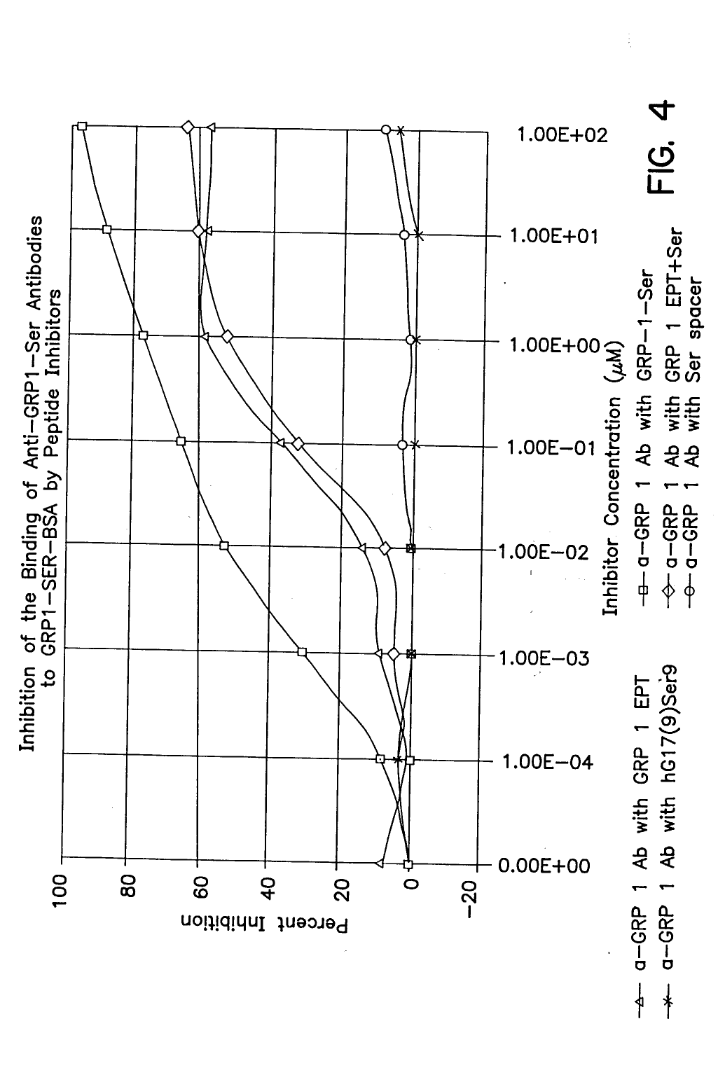

An inhibition ELISA was used to assess the specificity of the affinity purified antibodies for GRE1-Ser peptide. The assays were run as follows: GRE1-Ser-BSA conjugate was coated onto 96 well plates (Immulon U bottom) by overnight incubation of 50 μl of a 2 μg/ml solution of conjugate in glycine buffer (0.1M, pH=9.5) at 4° C. Affinity purified anti-GRE1 Ab (at a final concentration of 10 ng/ml) was combined with various inhibitors (in 1:10 dilution series) and incubated for 1 hour at room temperature. The inhibitors included GRE1-Ser, GRE1 EPT, Ser, human gastrin 17(1-9)-Ser spacer (hG17(9)-Ser), GRE1 EPT+Ser, and buffer (no inhibitor). Incubation buffer consisted of PBS+0.5% BSA+0.05% Tween 20+0.02% NaN₃. Subsequent steps used the same buffer without BSA. The 96 well plates were washed free of nonbound GRE1-Ser-BSA, and the Ab+inhibitor mixtures were added (50 μl/well). After 1 hour, the plates were washed and a goat anti-rabbit Ig (H+L) alkaline phosphatase conjugate (Zymed) was added (1:2000 dilution). After 1 hour incubation, the plates were washed to remove nonbound reagent, and 50 μl/well of pNPP substrate (Sigma) solution (1 mg/ml) was added in substrate buffer (PBS+0.1 mg/ml MgCl₂+10% diethanolamine+0.02% NaN₃). Following a 60 minute incubation, absorbance was measured on a MRX reader (Dynatech Laboratories). Samples were run in duplicate, and means were calculated for each concentration. Background binding (established with affinity purified rabbit anti-GnRH antibodies) was subtracted from all values, and the % Inhibition relative to no inhibitor added (anti-GRE1 Ab+buffer) was calculated for each inhibitor tested: % Inhibition=(100)(A_{uninhibited}−A_{inhibited})/A_{uninhibited}), where A=Absorbance. The results are shown in FIG. 4.

FIG. 4 presents the percent inhibition of antibody binding as a function of inhibitor concentration. As can be seen in the figure, the GRE1-Ser peptide fully inhibited antibody binding to GRE1-Ser-BSA. Approximately 60% inhibition was attained with the GRE1 EPT peptide, which does not contain the Ser spacer sequence, and by an equimolar mixture of GRE1 EPT plus Ser spacer. The failure of these peptides to produce full inhibition suggests that a proportion of the antibodies were specific for an epitope(s) comprising elements of both the GRE1 and the Ser spacer sequences. No inhibition was obtained by either the Ser spacer sequence itself or by an unrelated peptide bearing the Ser spacer (“hG17(9)-Ser”, consisting of the amino-terminal nine residues of hG17 followed by the Ser spacer). These ELISA results demonstrate that the affinity purified antibody preparation was specific for the GRE1-Ser peptide, and that 60% of the binding activity was directed against the gastrin-receptor epitope component of the peptide.

### EXAMPLE 3

AR42J tumor cells (European Collection of Animal Cell cultures, Porton Down, UK) are derived from a rat pancreatic adenocarcinoma and are known to have well characterized CCK-B/gastrin-receptors. Thus AR42J were tested to confirm the expression of GR and specificity of the receptor for hG17 by radioligand inhibition. AR42J cells were cultured at 37° C. with 7% CO₂ in complete RPMI 1640 (Sigma) supplemented with 10% FCS (Gemini Bioproducts), 2 mM glutamine (JRH Biosciences), 1 mM sodium pyruvate (JRH B.) and 50 μg/ml gentamicin (Gemini Bioproducts). The cells were harvested from 175 cm² T-flasks (Falcon Plastics) with PBS containing 0.25% EDTA, then washed twice with PBS (no EDTA) by centrifugation (400×g for 10 min). The cells were kept at 0-4° C. for all manipulations. A single cell suspension was prepared in buffer, and the cell concentration was adjusted to 10⁶ cells/ml. Aliquots of 1 ml of cell suspension were added to 12×75 mm culture tubes, then the cells were centrifuged and the supernatants discarded. The cells were resuspended in PBS (0.1 ml/tube) containing human G17 (hG17), gonadotropin releasing hormone (GnRH), or no peptide. The peptide concentrations were 1.0 ng/ml, 100 ng/ml and 10 μg/ml. An aliquot of 0.1 ml of ¹²⁵I-hG17 (NEN), containing approximately 26,300 CPM (specific activity, 2200 Ci/mmol), was added to each tube. The tubes were vortexed, then incubated for 15 minutes. The cells were washed twice with PBS, then counted in a γ counter (Wallac). Samples were run in duplicate. Background counts were subtracted, then the % inhibition of ¹²⁵I-hG17 binding by each inhibitor was calculated using the equation: % Inhibition=(100)(CPM_{uninhibited}−CPM_{inhibited})/CPM_{uninhibited}).

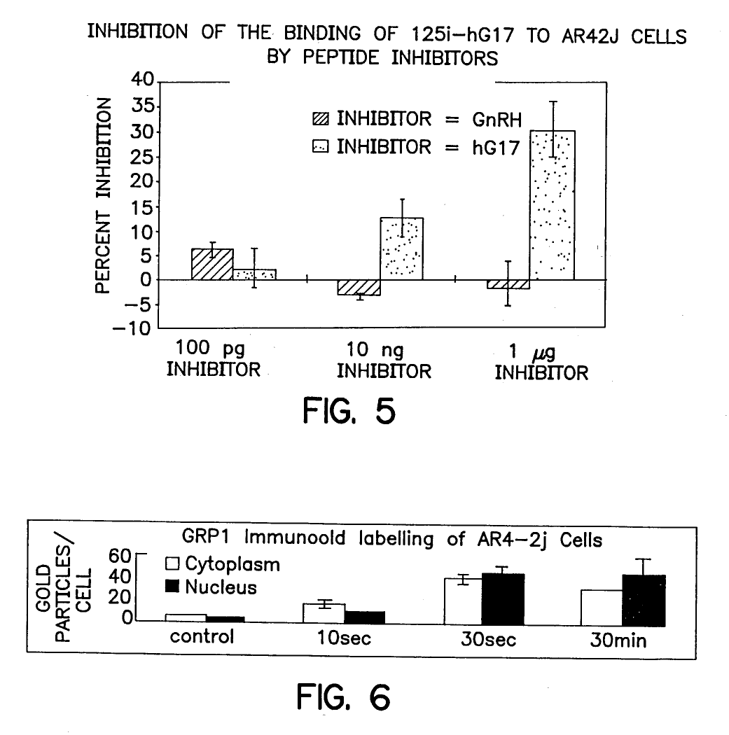

The results of the radioligand binding inhibition tests are shown in FIG. 5, which presents the means (±SE) of the individual values. As can be seen in the figure, binding of ¹²⁵I-hG17 to AR42J cells was inhibited by hG17. The degree of inhibition increased with the quantity of inhibitor added, to 32% inhibition at 1 μg hG17 per tube, the highest concentration of peptide tested. Conversely, GnRH produced no inhibition at the two highest concentrations tested (the 6% inhibition obtained with 100 pg GnRH was considered to be nonspecific), indicating that the inhibition by hG17 was specific for gastrin. These results confirmed the cell surface expression of gastrin-receptor by the AR42J tumor cells.

### EXAMPLE 4

Binding of the GRE1-Ser specific antibodies to AR42J cells was assessed by immunofluorescence. AR42J cells were grown as in the previous Examples and harvested with cell scrapers from 175 cm² T-flasks and washed twice with buffer (PBS with 0.02% NaN₃) by centrifugation (400×g for 7 min). The cells were kept at 0-4° C. for all manipulations. A single cell suspension was prepared in buffer, and the cell concentration was adjusted to 10⁶ cells/ml. The cell suspension was added to 1.5 ml microfuge tubes (1 ml/tube). The cells were pelleted by centrifugation and supernatants were aspirated. The cells were resuspended in buffer (0.1 ml/tube) containing peptide inhibitors (1.0 mg/ml). The inhibitors included GRE1-Ser, GnRH, hG17(9)-Ser and buffer (no inhibitor). Antibodies, including the rabbit anti-GRE1-Ser (100 μg/ml), affinity purified rabbit anti-DT (negative control, 100 μg/ml), mouse anti-AR42J antiserum (positive control, 1:100 dilution, heat inactivated) or normal mouse serum were added to the appropriate tubes and the contents were mixed. The cells were incubated for 1 hour, with occasional mixing. The cells were then washed three times with buffer, and 0.1 ml of fluorescein-labeled goat anti-rabbit IgG (Antibodies Incorporated) (diluted 1:50) was added per tube. The cells treated with mouse sera were developed with a fluorescein-anti-mouse IgG reagent (Zymed). The cells were re-suspended by vortexing, then incubated for 1 hour. The cells were again washed three times, then re-suspended in glycerol:PBS (1:1, v:v), 50 μl/tube. Wet mounts were prepared with the contents of each tube, and the cells examined using a Laborlux 12 fluorescent microscope (Leitz). Fluorescence was scored on a scale of 0 to 4, with 0 representing background fluorescence (obtained with the normal mouse serum) and 4 representing maximal fluorescence (obtained with the mouse anti-AR42J positive control antiserum).

The results of the immunofluorescesce tests are presented in Table 2. As can be seen in the Table, AR42J cells treated with anti-GRE1-Ser antibodies in the absence of peptide inhibitors fluoresced strongly, indicating that the antibody bound to the cells. Rabbit anti-DT antibodies did not produce fluorescent staining, demonstrating that the staining observed with the anti-GRE1-Ser antibodies was not a consequence of non-specific cell surface binding by rabbit immunoglobulin. Moreover, the binding was shown to be specific for the GRE1-Ser peptide. Addition of GRE1-Ser fully inhibited binding, whereas unrelated peptides, including hG17(9)-Ser and GnRH, failed to inhibit. As the GRE1 epitope comprises residues 5-21 of the gastrin-receptor sequence, it was concluded that the anti-GRE1-Ser antibodies were specific for the gastrin-receptor expressed by AR42J cells.

TABLE 2

| Antibody | Inhibitor | | | |
|---|---|---|---|---|
| Preparation | GRE1-Ser | hG17(9)-Ser | GnRH | Buffer |
| Rabbit anti-GRE1-Ser | 0 | 3+ | 2+ | 3+ |
| Rabbit anti-DT | 0.5+ | 0.5+ | 0.5+ | 0.5+ |
| Mouse anti-AR42J | | | | 4+ |
| Normal Mouse Serum | | | | 0 |

### EXAMPLE 5

AR42J cells, passage nos. 16-18 were cultured in RPMI-1640 medium containing 10% FCS and 2 mM glutamine. All cells were maintained at 37° C. in 5% CO₂ in air at 100% humidity, grown to 80% conflucency in T75 flasks (Falcon, London, UK) and passaged following a 0.02% EDTA treatment to bring adherent cells into suspension. Cells were incubated for 10, 30 seconds, 30 minutes and 1 hour with anti-CCK-B/gastrin-receptor antibody (aGR) generated in rabbits with a CCK-B/gastrin Peptide 1 receptor immunogen of the invention as described in Example 1, which had been purified by affinity chromatography in a column prepared with Peptide 1.

The cells were fixed in 1% glutaraldehyde for one hour and prepared for immunoelectron microscopy (ImmunoEM) studies using standard techniques. The cell suspensions was centrifuged twice at 2000 rpm for 2 minutes and then the cell pellet resuspended in phosphate buffered saline (PBS). The cell pellet was infiltrated with LRwhite plastic resin. Ultrathin sections of 70-90 nm in thickness were cut and place on Pioloform coated nickel grids. The grids were placed in normal goat serum (Dako, High Wycombe, UK) in 0.1% bovine serum albumin (BSA) (Sigma, Poole, Dorset) and incubated at room temperature for 30 minutes. Grids were rinsed in PBS then incubated with a secondary antibody, gold particle-labeled goat anti-rabbit antibody, diluted 1:50 in 1% BSA, for 1 hour at room temperature. Control experiments were performed without secondary antibody. After final PBS wash, the grids were counterstained in saturated aqueous uranyl acetate for 3 minutes and Reynold's lead citrate for 3 minutes. Gold particles on the cell membrane, in the cytoplasm, on the nuclear membrane and within the nucleus were counted. Twenty-five cells/grid were counted by an independent observer. For controls AR42J cells were exposed to antibodies for less than 1 second, and liver cells which are devoid of GR were used. AR42J cells exposed to normal IgG were also used as controls for determining non-specific binding of the anti-GR antibodies. The results of these experiments are shown in Table 3 and FIG. 6.

TABLE 3
Distribution of CCK-B/gastrin-receptor Immunogold
Particles Within AR42J cells

| | Cell | | Nuclear | Nuclear |
|---|---|---|---|---|
| | membrane | Cell matrix | membrane | matrix |
| No. gold particles | 14.2(±0.97) | 43.3(±2.32) | 9.3(±0.81) | 51.4(±3.32) |
| Percent | 12% | 36.6% | 7.9% | 43.5% |
| distribution | | | | |
| within cell | | | | |

(mean ± SEM for 25 cells, repeated n = 5.)

As demonstrated in Table 3 and FIG. 6, immunogold-antibody particles attached to the GR were localized on plasma membrane, cytoplasm, nuclear membrane, and nuclear matrix of the adenocarcinoma cells, further demonstrating that the antibody/receptor complex is internalized by the cells.

As seen in Table 3, the immunoEM studies using an antiserum directed against the amino-terminal end of the GR shows that after one hour incubation, the distribution of immunogold-labelled GR antibody is quickly internalized as 12% of the antibody receptor complex is associated with the cell membrane, 36.6% is within the cytoplasm, 7.9% is in the nuclear membrane and, quite surprisingly, 43.5% is within the cell nucleus. Areas of intense GR immunoreactivity within the nucleus are found on chromatin, which may suggest specific binding sites for regulation of the DNA.

These electron microscopy studies with anti-immunoglobulin conjugated to gold beads (immmunogold) reveal a rapid turnover of the anti-receptor antibody/receptor complex in the tumor cells; as seen in FIG. 6. These tests also demonstrate that the anti-GR antibodies are taken into the nucleus of tumor cells.

### EXAMPLE 6

Adenocarcinoma cell lines, namely AR42J, HCT116, C170HM2, LoVo, ST16 and MGLVA1, were grown in vitro and harvested as described in Examples 3. Cells from thirty T-75 flasks were suspended in 5 ml of homogenization buffer (1 mM sodium hydrogen carbonate, 2 mM magnesium chloride, 1 nM phenylmethylsulfonyl fluoride, 40 mM sodium chloride, 10 μl leupeptin, 1 μM pepstatin, 5 nM EDTA [Sigma]). Homogenization was carried out by 5 bursts of 5 second duration in a homogenizer. For extranuclear membranes, tissue debris was pelleted by centrifugation at 500 g, 7 minutes, 4° C. The pellet was discarded and the supernatant centrifuged at 500 g, 4° C. to remove further debris. The supernatant was recentrifuged at 48,000 g, 1 hour, 4° C. The pellet containing the extranuclear membrane preparation was suspended in Tris/NP-40 solution (0.1M TRIZMA, 0.5% NONIDET P40 [Sigma Chemical]).

For nuclear membrane preparations, following homogenization in a second homogenization buffer (25 mM Tris-HCl, pH 7.4, 0.1% TRITON 100, 0.32 M sucrose, 3 mM MgCl₂, 2 mM EGTA, 0.1 mM spermine tetrahydrochloride, 2 mM PMSF, 10 mM bezomidine hydrochloride, 3 mM EGTA aminoacetonitrile hydrochloride [Sigma]), tissue debris was pelleted by centrifugation at 400 g, for 10 minutes at 4° C. The pellet was resuspended in 55% (0.2 M) sucrose in HPLC water. This mixture was spun at 60,000 g for 1 hour at 4° C. The pellet was washed with 0.4% NONIDET P40 in homogenization buffer without TRITON 100. The pellet was spun at 700 g for 15 min at 4° C. and resuspended in homogenization buffer without TRITON 100.

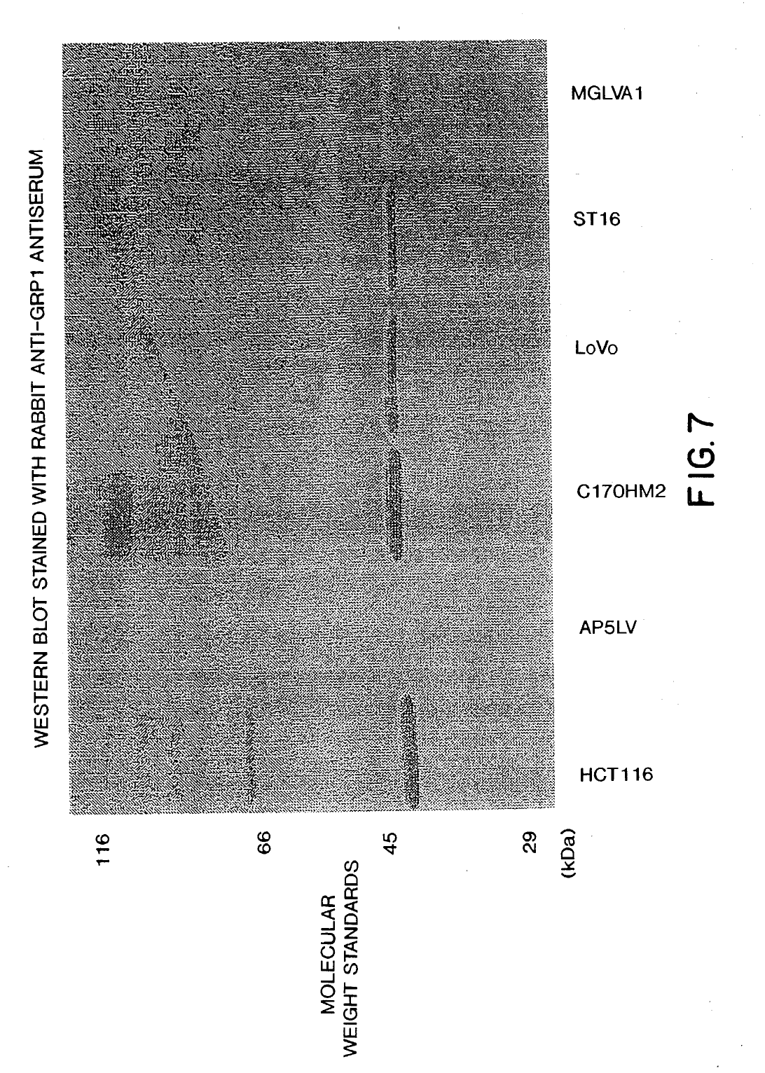

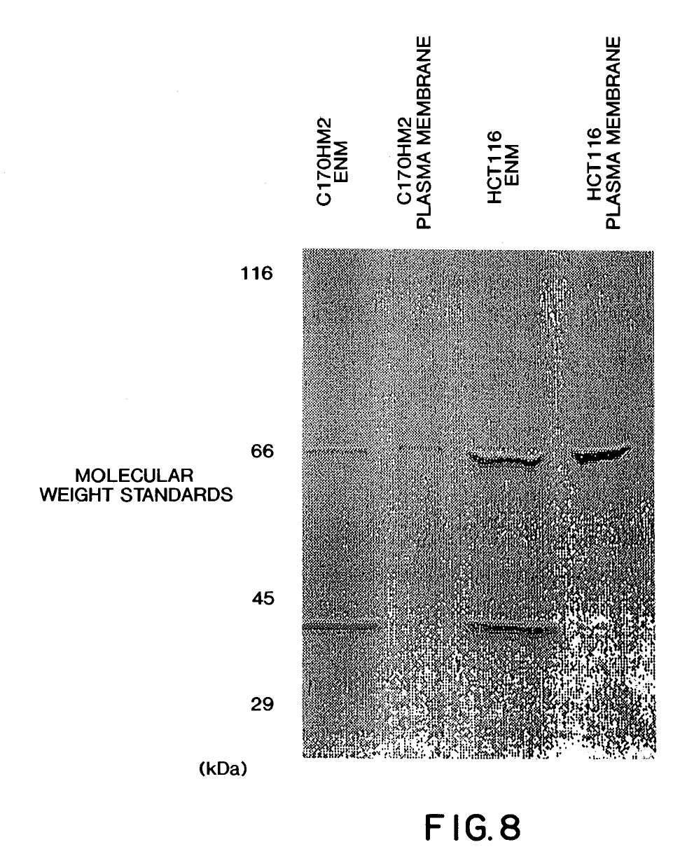

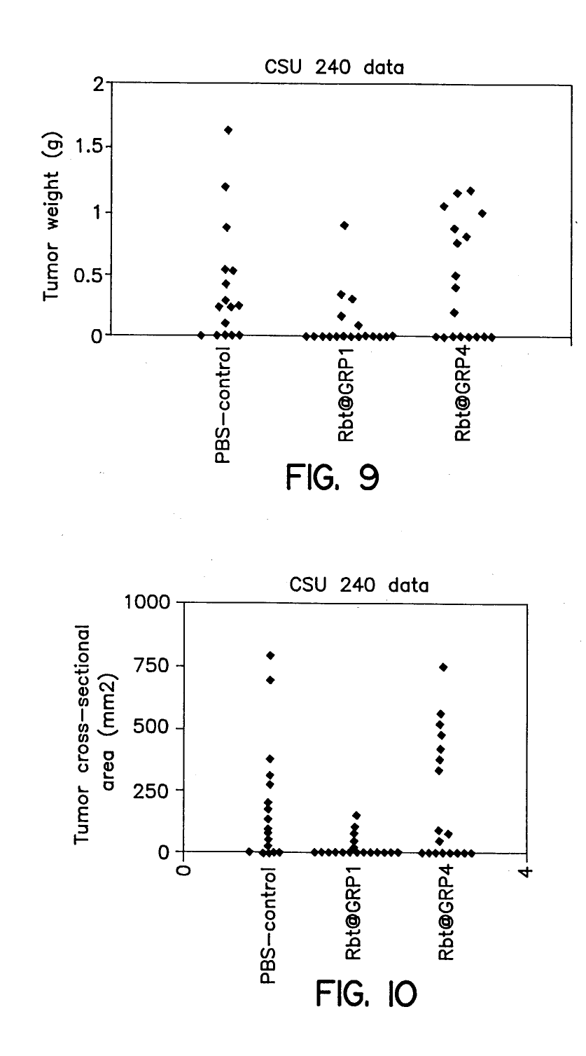

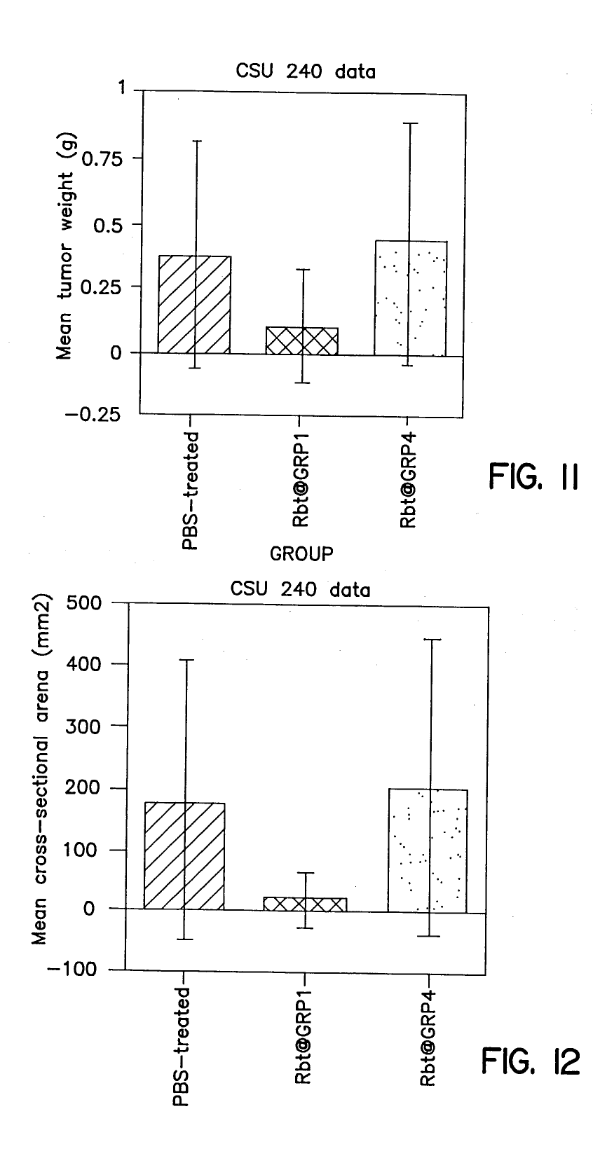

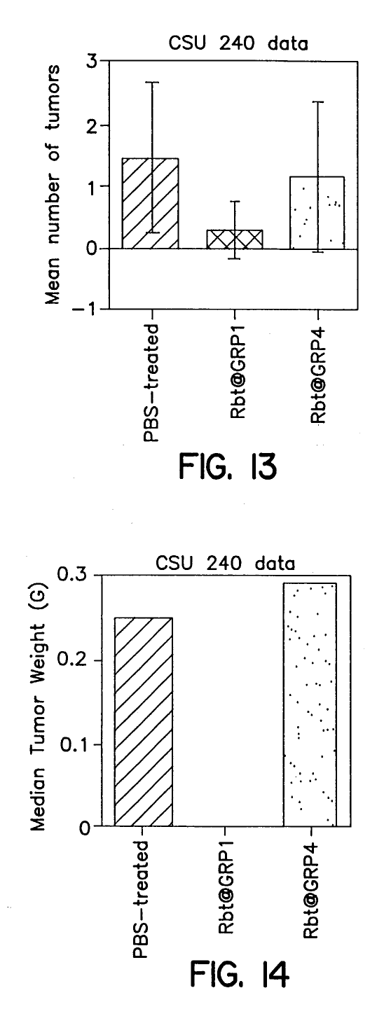

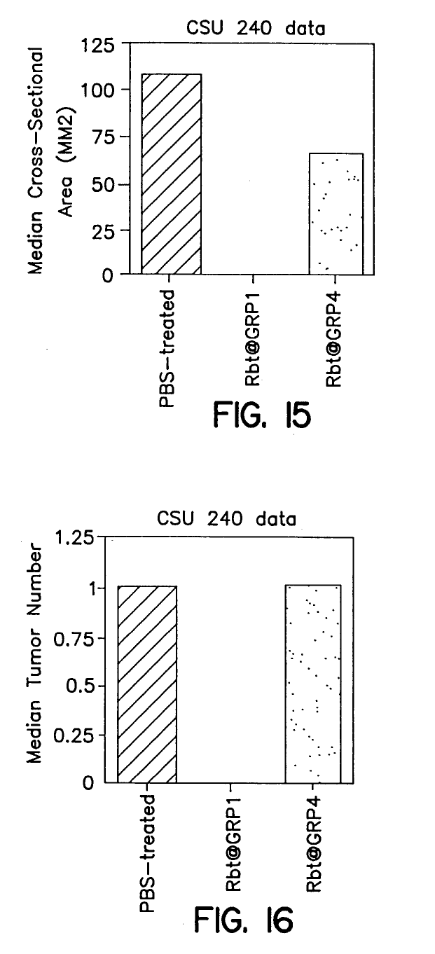

Protein content was determined by the Lowry method (using a kit from Pierce). Samples containing 10-15 μg of protein were loaded onto a 8-16% Tris/glycine gradient polyacrylamide gel electrophoresis PAGE (Novex R and D systems) in Tris/glycine buffer and run for 90 minutes at 125 constant volts, 36 mA. The gel was fixed in 10% glacial acetic acid for 1 hour and samples were blotted onto nitrocellulose membrane. The membranes were incubated in 1% BSA for 1 hour, followed by incubation with GRE1 antiserum (with and without preabsorption) for 1 hour. Antibody binding were detected by the avidin:biotin-peroxidase complex method using diamino-bezidene as the substrate. The Western blot analysis results using Rabbit-antiserum raised against GRE1 (Rabbit anti-GRE1 antiserum) are shown in FIG. 7 and FIG. 8.

As shown in FIG. 7, the protein molecular weight markers included proteins of 116, 66, 45 and 29 kDa. The blot shows a prominent anti-GRE1 immunoreactive band localizing at about 43 kDa in all adenocarcinoma cells studied, i.e., HCT116, C170HM2, LoVo, ST16 and MGLVA1, except one (AP5LV). This protein corresponds to a truncated form of the GR. Some cell lines (HCT 116 and C170HM2) show at least 3 other bands, ranging in molecular weight between 60 and 100 KDa. The data indicate that the anti-CCK-B/gastrin-receptor antibodies can recognize and bind to various isoforms of the CCK-B/gastrin-receptor in tumor cells.

FIG. 8 shows a Western blot from extranuclear (ENM) and plasma membrane of C170HM2 and HCT116 adenocarcinoma cells. As shown in FIG. 8, adenocarcinoma cell lines tested for ENM GR demonstrate the existence of two strongly stained bands: one about 43 KDa and the other at about 66 KDa. When only the plasma membrane fraction was stained, a single band at about 66 KDa was present. Thus, the Western blot studies confirm the immunoEM results that the GR is present in adenocarcinoma tumor cells, although the immunoEM studies do not distinguish between the isoforms of the GR. The data indicate that the present immunogens elicit anti-GR antibodies which can recognize and bind various isoforms of the receptor, which would be advantageous for the treatment of these tumors.

### EXAMPLE 7

To detect expression of CCK-B/gastrin receptor in theadenocarinoma cell lines, RT-PCR was performed to detect CCK-B/gastrin receptor mRNA. Total RNA was isolated from all cell lines. Cell suspensions were prepared using trypsin-EDTA, and total RNA isolated from 1-3×10⁶ cells using the SV total RNA isolation system (Promega) according to the manufacturers directions. Reverse transcription and PCR were carried out using the one-step Access RT-PCR system (Promega), using specific primers for both gastrin receptor (McWilliams et al. 1998) and β actin (10) as a positive control. RT was carried out at 48° C. for 45 minutes; PCR was 40 cycles of 94° C. for 45 s, 60° C. for 90 s, 68° C. for 2 min.; a double round was performed for gastrin receptor mRNA amplification. Products were analysed by agarose gel electrophoresis.

RT-PCR performed on all cell lines confirmed the presence of gastrin receptor mRNA in AR42J, C170HM2, HepG2 and NIH3T3 cells transfected with the classical and truncated forms of the gastrin receptor gene. Gastrin receptor mRNA was not detected in non-transfected NIH3T3 cells. All cell lines were positive for β actin mRNA (see Table 4).

TABLE 4
RT-PCR for β-actin and gastrin receptor for mRNA on cell lines.

| | β actin | |
|---|---|---|
| Cell Line | amplification | Gastrin receptor amplification |
| NIH3T3 transfected (long) | + | + |
| NIH3T3 transfected (short) | + | + |
| NIH3T3 non-transfected | + | − |
| AR42J | + | + |
| HepG2 | + | + |
| C170HM2 | + | + |

The results showed AR42J, C170HM2, HepG2, transfected NIH3T3 positive for β actin and gastrin receptor mRNA and non-transfected NIH3T3 cells as positive for β actin mRNA only.

Uptake of RG-G7

Binding and internalization of rhodal green labeled heptagastrin (RG-G7) was seen in AR42J cells, HepG2 and C170HM2 cells. Gastrin was taken up into the cytoplasm of these cells. Binding was also seen in NIH3T3 cells stably transfected with gastrin receptor, but not in non-transfected NIH3T3. In the transfected cells, gastrin appeared to be bound to the cell membrane. Fixed AR42J cells showing gastrin uptake were stained for gastrin receptor using Alexa-546 conjugated GRE1 antibody—coexpression of gastrin and gastrin receptor was seen. Gastrin receptor was detected on the membrane and within the cytoplasm of these cells.

No binding or internalization of RG-G7 was seen in non-transfected NIH3T3 cells.

Confocal photomicrograph was performed of AR42J cells which were first incubated with RG-G7 (green), then fixed and stained with anti-gastrin receptor antibody, GRE1. It was observed that Gastrin was taken up into the cytoplasm of these cells, such that co-localization of gastrin and gastrin receptor could be seen; gastrin receptor alone could be seen on the surface of one cell. Optical sectioning of the cells using the confocal microscope confirmed that gastrin was taken up into the cytoplasm but not the nucleus of cells.

Uptake of Anti GRE1 Antibodies

In the tumour cell lines AR42J, C170HM2 and HepG2, addition of anti GRE1 antibody to live tumor cells resulted in binding and internalization of the antibody into the cytoplasm and the nucleus of cells. F(ab) and F(ab)₂ fragments of anti GRE1 antibody were also incubated with live cells from these tumor cell lines, and uptake into the cytoplasm and nucleus was seen. No uptake of FITC—conjugated irrelevant antibodies (rabbit anti-mouse Ig, F(ab)₂ fragment) by these cell lines was seen. Uptake of anti-GRE1 antibody was not seen in non-transfected NIH3T3 cells, or in normal lymphocytes, but was seen in NIH3T3 cells transfected with the wild type and truncated forms of the human gastrin/CCK-B receptor. Anti-GRE1 Antibody appeared to bind to the membrane of transfected NIH3T3 cells, but was not taken up into the cytoplasm or the nucleus of these cells. This was a different pattern of uptake from that seen in the tumor cell lines that normally exposes the GR.

AR42J cells incubated with Alexa-546 labelled anti-human GRE1 antibody showed uptake of the antibody into the cell. Optical sectioning of the cell using the confocal showed antibody uptake into the nucleus as well as the cytoplasm of the cell.

Anti-GRE1 antibody added to a) C170HM2 cells and b) HepG2 cells can be seen within the cytoplasm and the nucleus of cells.

The specificity and sensitivity of immunostaining obtained using GRE1 antibody labelled with Alexa-546 was confirmed by staining sections from a formalin fixed paraffin embedded (FFPE) gastrinoma. Nuclear staining of the gastrinoma was obtained with both fluorescently labelled and unlabelled GRE1 antibody; this staining could be abolished by preabsorbance with epitope. F(ab) and F(ab)₂ fragments of GRE1 labelled with rhodamine gave the same staining pattern on this material; this staining could also be abolished by preabsorbance of the antibody with the epitope.

Gastrinoma (FFPE) stained with anti-gastrin receptor antibody GRE1; a) binding detected by anti-rabbit^{AP} (red), showing nuclear staining; b) Alexa-546 labelled GRE1 (red fluorescence) showing nuclear staining of gastrinoma and no staining of background liver; c) Alexa-546 labelled GRE1 after epitope preabsorbtion of the antibody, abolishing staining.

Addition of Fab¹ and F(ab)₂ fragments of GRE1 antibody to live tumour cells again show GRE1 within the cytoplasm and nucleus of AR42I cells, HepG2 cells and C17OHM2 cells.

Accumulation of anti-GRE1 Ab within cells over time was studied to assess how quickly antibody is taken up into different cell compartments. AR42I cells were used for this series of experiments; cells were incubated with antibody at 37° C. or 4° C. and observed after varying periods of time, up to 1 hour. In cells incubated at 37° C., anti-GRE1 Ab was observed within the nucleus after only 5 minutes incubation. Binding of antibody to the membrane and translocation across the cell was too rapid to be observed. After 15 minutes at 4° C., the antibody had been taken up into the cytoplasm and nucleus of some cells.

Thus, the experiment demonstrated the internalization of gastrin and gastrin receptor in tumour cell lines of colonic, pancreatic and hepatic origin, and also in NIH3T3 cells transfected with classical and truncated isomers of human GR.

Internalization of gastrin and anti-gastrin receptor antibody was seen only in cells containing gastrin receptor mRNA, and not in non-transfected NIH3T3 cells or in normal lymphocytes, not expressing CCK-B receptor or actively expressing GR mRNA.

Confocal microscopy has confirmed that anti-gastrin receptor antibody is internalized by tumor cells and can be detected within the cytoplasm and the nucleus of cells. This internalization is rapid and specific; the anti-GRE1 Ab was seen within the nucleus of AR42J cells after 5 minutes incubation at 37° C. Internalization was observed furthermore with antibodies produced by other anti-amino terminal GR peptides, such as GRE-11.

Internalization is not mediated via the Fc receptor as F(ab) fragments of GRE1 antibody were internalized in a similar way, and no internalization of irrelevant antibodies was detected under identical conditions. Internalization is therefore specific.

### EXAMPLE 8

C170HM2 adenocarcinoma cells were injected intraperitoneally into nude mice and tumors were allowed to grow in the liver. Control mice received an infusion of phosphate buffer saline solution (PBS) and experimental mice received an infusion of the anti-GR antibodies. In Group 1, each mouse was infused daily with 0.5 mg of Rabbit anti-GR antibodies generated against one of the peptide epitopes, i.e. Rabbit anti-GRE1. In Group 2, each mouse received daily 0.5 mg of Rabbit anti-GR antibodies generated against GRE4, i.e. Rabbit anti-GRE4. The mice were studied for a period of 40 days after antibody infusion, sacrificed and the tumors removed for study. The weight, size and cross-sectional area of the tumors were assessed by standard techniques.

Implantation of the colorectal adenocarcinoma cancer cell line C170HM2 in mice without treatment resulted in the rapid growth of large tumor masses, as determined by tumor weight, or tumor size, and the tumor cross-sectional area of the tumors. However, infusion of the animals with Rabbit anti-GRE1 or Rabbit anti-GRE4 antibodies resulted in a marked decrease in the number of animals having any detectable tumor, as well as in the weight and size of tumors in animals having them when compared to controls. The same effect can be seen when mean tumor weight, mean tumor size, or mean tumor number is calculated.

Further insight into the distribution within the population is gained by calculating the medians of tumor numbers, weight and size. The Rabbit anti-GRE1 antibodies were consistently more effective than Rabbit anti-GRE4 antibodies in inhibiting tumor growth. However, both Rabbit anti-GRE1 and Rabbit anti-GRE4 antibodies did exhibit powerful tumor inhibitory activity as compared to the control treatment. In addition, in another study, Rabbit anti-GRE11S was found to be at least as effective as Rabbit anti-GRE1, as shown below.

### EXAMPLE 9

A larger tumor burden was generated in nude mice using the colon cancer cell line C170HM2 by a method as described in Example 8, but with a higher initial cell innoculum. The C170HM2 is a liver-invasive xenograft model. Control and experimental mice were treated also as described in Example 8.

Forty days after antibody infusion, the mice were sacrificed and liver tumors were removed and studied. FIGS. 17, 18 and 19 show the results of these experiments. FIG. 17 shows the mean and median numbers of liver tumors in control and anti-GR antibody treated animals. The data show that the rabbit-anti-GR antibodies (“Rabbit@GRE”) are effective in inhibiting the growth of the metastatic tumors in the liver. There is a statistically significant (p<0.05) decrease in mean liver tumor numbers in mouse livers using Rabbit anti-GRE1 (Student's T test), p=0.0084 and in the median liver tumor number, p=0.0016 (Mann Whitney) when compared to controls. Mice treated with anti-GRE4 antibodies also show a decrease in mean liver tumor number; however, there was no difference in the mean liver tumor number in these animals when compared to controls.

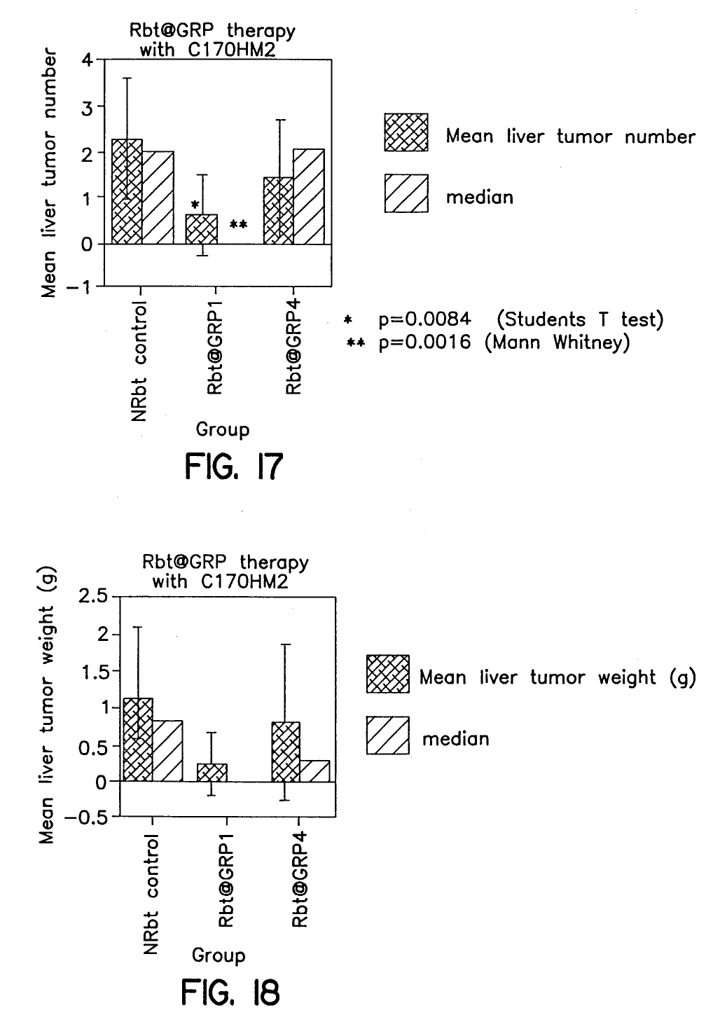

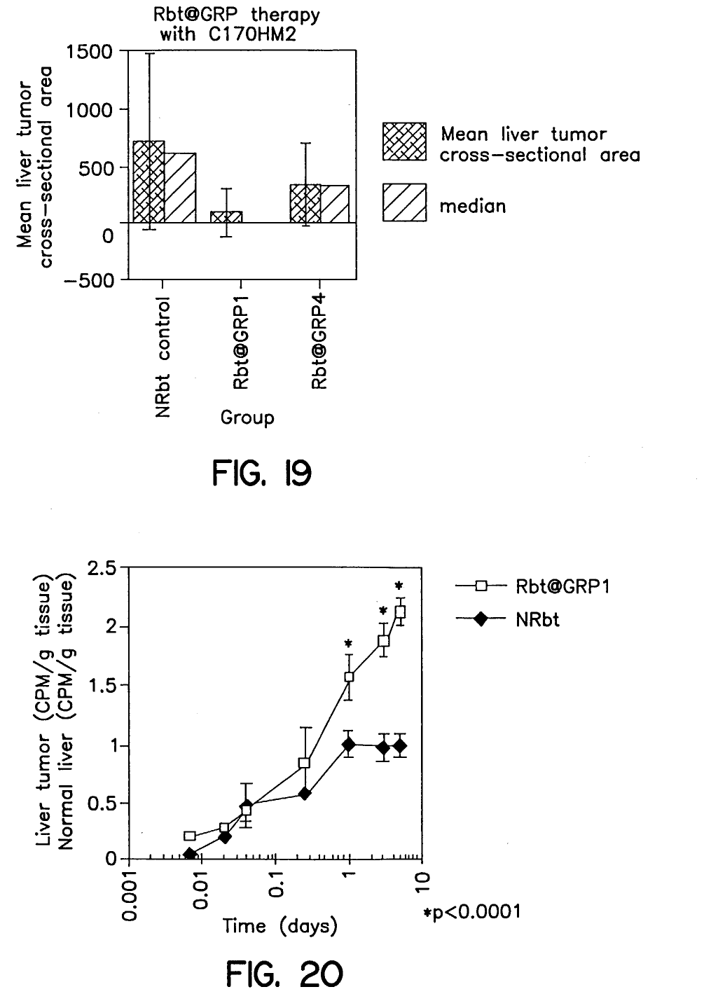

FIG. 18 shows that anti-GRE1 and anti-GRE4 antibodies were also capable of reducing the mean and median tumor weights of liver metastases when compared to control animals. The data in FIG. 19 show that anti-GR treated mice also had a significant decrease in mean and median cross-sectional area of the liver tumors when compared to control animals.

The data indicate that the anti-GR antibodies are effective in controlling the spread and growth of a gastrin-dependent colon cancer in the liver, which constitutes the major site of metastatic spread of this cancer.

### EXAMPLE 10

These studies we carried out to confirm GRE1 immunoreactivity on C170HM2 cells. The aim of the study was to evaluate tumor localization of antiserum raised against GRE1 and to determine its therapeutic effect on the growth of C170HM2 cells within the liver of nude mice. C170HM2 cells were injected intraperitoneally into nude mice as described in Examples 8 and 9 above. GRE1 antiserum was raised in rabbits. The antiserum was radiolabelled with ¹²⁵I and administered to nude mice with established C170HM2 xenografts by a tail vein injection. Control mice received ¹²⁵I radiolabelled normal rabbit serum. Mice were terminated at increasing time points following injection of a single dose of ¹²⁵I antibodies. Radioactivity was measured as counts per minute per gram of (CPM/g) tissue and the liver/liver tumor ratio calculated.

FIG. 20 is a graph which shows the radiolabeled rabbit anti-GRE1 antibodies bound to liver tumors versus control. As seen in the figure, more rabbit anti-GRE1 antibodies are bound to liver tumor tissue when compared to controls. FIG. 20 also shows the liver tumor/liver ratio on the y axis with increasing time on the x axis for both radiolabeled normal rabbit serum and anti-GRE1 antiserum. The normal rabbit serum achieved a ratio of 1 from day 1 which remained constant until day 5. This indicates the level of radiolabel in the liver tumour and normal liver was equal. The ratio for GRE1 antiserum accumulated exponentially approaching 2 by day 5. This indicates radiolabeled GRE1 antiserum specifically localizes within C170HM2 liver tumors. Thus, radiolabeled GRE1 antibodies could be used for diagnostic imaging of tumor or for radioimmunotherapy of tumors, depending upon the radionuclide coupled to the antibody.

### EXAMPLE 11

Therapeutic Effect of GRE1 Antiserum on C170HM2 Xenografts

The C170HM2 tumor xenografts were initiated by intraperitoneal injections of cells. Three different cell inocula were used to generate 3 levels of tumor burden. The GRE1 antiserum was administered passively by tail vein injection daily from day 0. Therapy was terminated on day 40.

Effect of GRE1 Antiserum on Tumor ‘Take Rate’

The initial parameter evaluated was mean tumor number within the liver which is shown in FIG. 21. The normal rabbit antiserum treated controls are grouped in increasing cell inocula. As seen in FIG. 21, in the control groups the mean tumor number per liver was between 1 and 3. In the GRE1 antiserum treated group the mean tumor number per liver was less than 1 for all three cell inocula, which was significant for all 3 experiments (one inoculum, n=18, p=0.003; 2 inocula, n=12, p=0.0001 and 3 inocula, n=20, p=0.0068, Mann Whitney analysis).

Effect of GRE1 Antiserum on Tumor Weight of Established Tumors

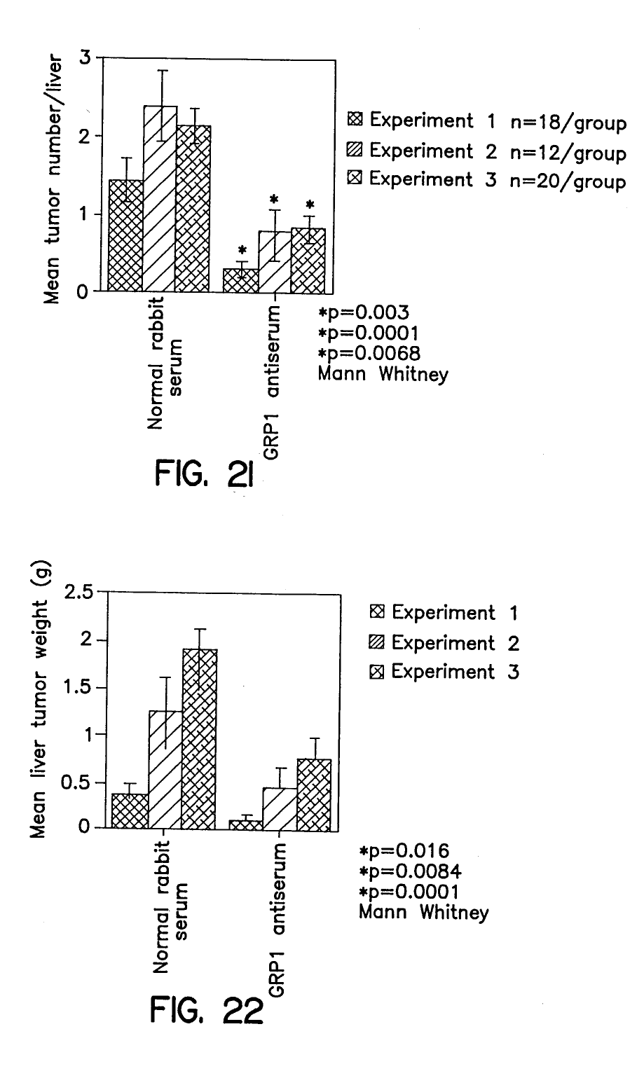

FIG. 22 shows the mean tumor weight for the normal rabbit serum treated controls on the left panel for the 3 increasing cell innocula. The figure also shows the mean tumor weight of nude mice following treatment with GRE1 antiserum. The mean liver tumor weight was reduced by 60% with all 3 cell innocula, which was significant for all 3 experiments (one inoculum, p=0.0016; 2 innocula, p=0.0084, and 3 innocula, p=0.0001, Mann Whitney analysis).

GRE1 Immunoreactivity in C170HM2 Xenografts as Determined by Western Blotting

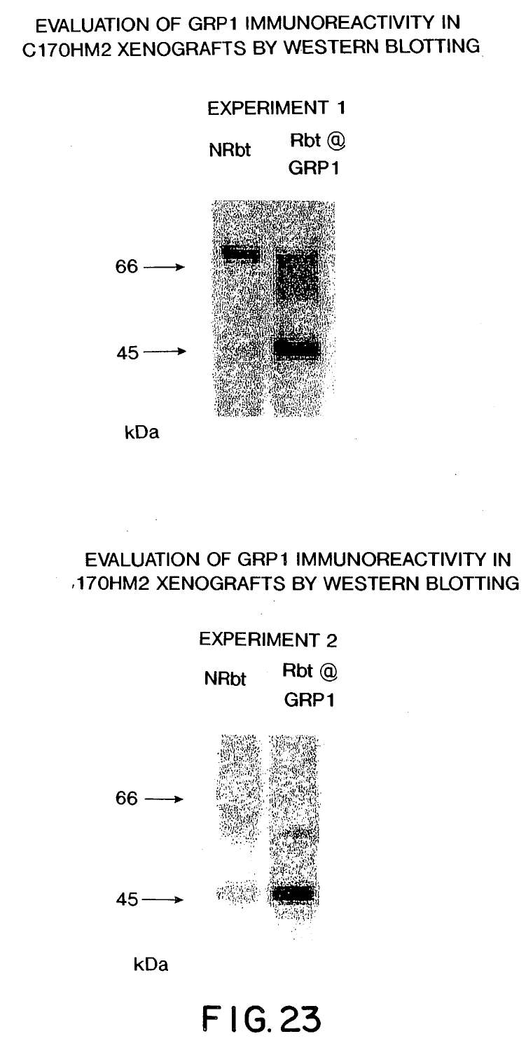

Extra-nuclear membrane proteins were prepared from C170HM2 xenografts from 2/3 experiments. These were analyzed by Western blotting using the GRE1 antiserum. FIG. 23 is a photograph of the Western blot showing that, in the normal rabbit serum-treated xenografts, two immuno-reactive bands were present at 74 and 50 kDa, with the former band showing the strongest immunoreactivity. In the GRE1 antiserum treated xenografts, there are 2 immuno-reactive bands together with an intermediate band, not seen in the control xenografts or cells grown in vitro. A 50 kDa band shows the strongest immunoreactivity. This indicates that in the GRE1 antiserum treated xenografts a larger proportion of the GR's may be present as an internalized form.

Histological Analysis of C170HM2 Xenografts

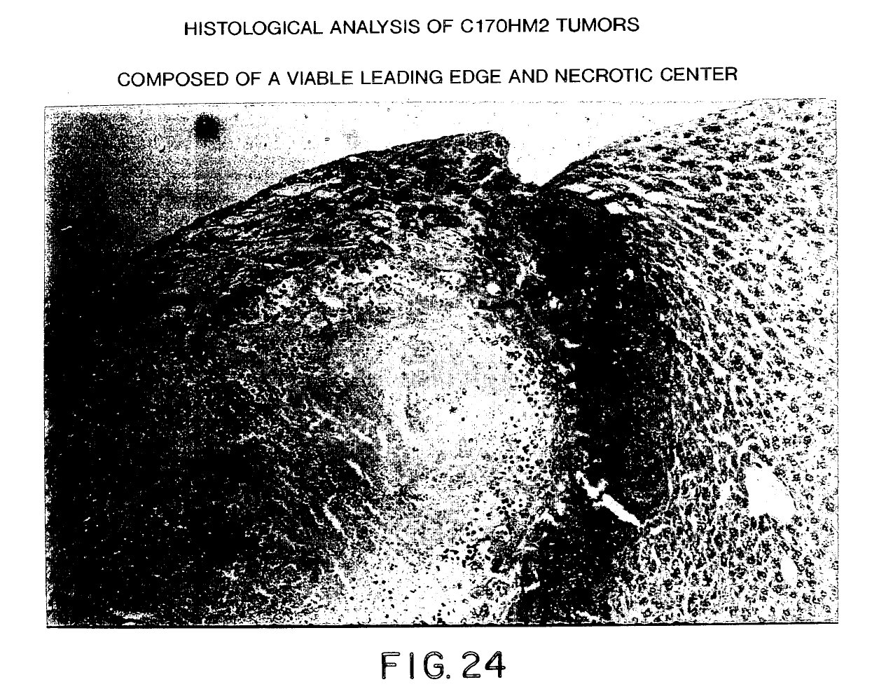

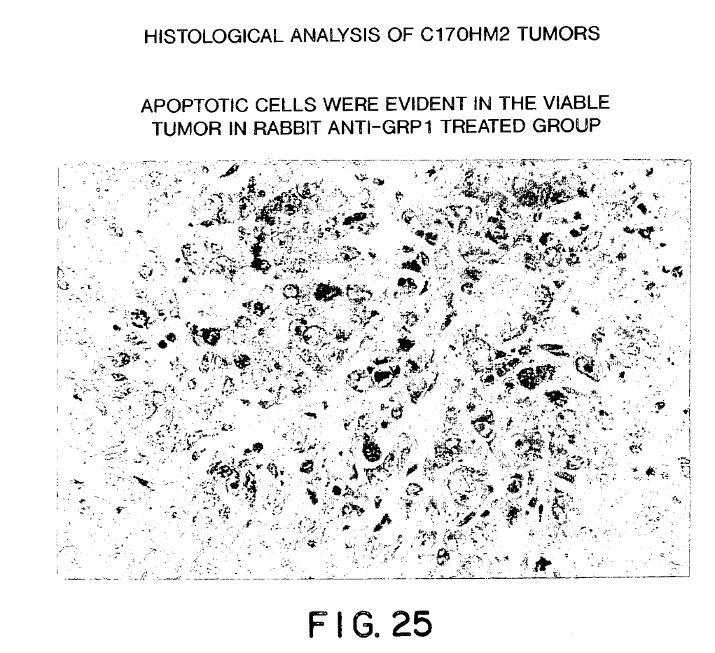

FIG. 24 shows a microscopic view of a C170HM2 xenograft invading a liver of a nude mouse. The tumor is generally composed of a necrotic center with a viable leading edge which squashes the hepatocytes as it invades the liver. The degree of apoptosis was measured in the viable leading edge of C170HM2 tumors by the Tunel method with positive cells visualized by in situ hybridization. FIG. 25 shows that apoptotic cells were present in the viable tumor cells in the GRE1 antiserum-treated xenografts, but not in the normal rabbit serum-treated tumors.

The data show that antiserum raised against the amino terminal epitope of the CCKB/gastrin-receptor selectively localizes within liver-invasive C170HM2 tumors. Neutralization of the GRE1 epitope induced a significant effect on both tumor ‘take rate’ and gross tumor burden of tumors that did establish. This tumor-inhibitory effect may be due to (a) a general cytostatic effect induced by blocking the GR and/or (b) an indirect effect of targeting an antibody to the nucleus of the cell, possibly resulting in apoptosis.

### EXAMPLE 12

The reverse synthetic peptide sequence immunomimic of the GRE-1 epitope (SEQ ID) NO: 5) of the human CCK-B/gastrin receptor (GR) has been linked through an N-terminal spacer peptide to an immunogenic carrier protein. Specifically, the synthetic amino acid sequence comprises CGG KLNRSVQGTGPGPGASL (SEQ ID NO: 5) the underlined portion represents the spacer sequence, the rest represents the N-terminal portion; 5-21 aa, of the CCK-B/gastrin 7-loop receptor.

The CGG-(5-21) peptide immunogen has been tested in suitable test animals, and the immune response has been measured.

### EXAMPLE 13

Another synthetic GR immunomimic, GRE11 (SEQ ID NO: 11), comprises amino acids 1-22 of the GR peptide sequence which is linked to an immunogenic carrier through the Cys residue located at position 22 of the native sequence of the peptide. Its sequence is as follows:

MELLKLNRSVQGTGPGPGASLC (SEQ ID NO: 11)

The GRE11 epitope encompasses also the GRE1 (5-21) epitope.

An immunogenic construct comprises the GRE11 peptide conjugated to an immunogenic carrier protein, such as DT. Another embodiment comprises the modified GRE11Ser spacer MELLKLNRSVQGTGPGPGASLSSPPPPC (SEQ ID NO: 12).

The immunized test rabbits showed induction of anti-GRE11 peptide antibodies.

Tests showed in vitro binding of the anti-GRE11 antibodies to the epitope GRE11, as partially inhibited by GRE-1. A quantitative immunofluorescence assay test showed much less cross-reactivity with the GRE6 epitope (1-12 aa). The GRE6 peptide has the N-terminal sequence:

MELLKLNRSVQG (SEQ ID NO: 8)

The quantitative immunofluorescence assay is read on a 96 well-fluorometer.

Furthermore, standard immunofluorescence and confocal microscopy was used to show uptake of the fluorescent labeled anti-GRE11 antibodies into live cells in culture.

It was found that anti-GRE11 antibodies are taken into the cytoplasm and into the cell nucleus. It was further discovered that the antibodies relocated in the cell's cytoplasm and/or nucleus produced or induced a suicidal process (i.e. apoptosis).

Western blotting identified anti-GRE11 antibody binding on GRE1 bands of GR+ cell extracts.

The antibodies are also tested by affinity purifying the GR+ cell extract over a ligand (i.e. Gastrin) affinity column. The purified GR is then probed in a Western blot against the GRE11 antibodies. It was found that the bands are identical with those of the cell extracts.

The GR identity is also tested by amino acid analysis and amino terminal sequencing of cut-out blotted bands.

As shown below, the anti-GRE11 antibodies have been shown in vitro to inhibit the proliferation of GRE+AR42J cells modified from a rat pancreatic cancer line, in comparison to anti-DT antibody controls.

The anti-GRE11 antibodies were also shown to induce apoptosis of GRE+ tumor cell line, in vitro.

### EXAMPLE 14

Quantitative antibody binding to cells expressing the gastrin receptor (GR) was demonstrated and challenged with synthetic GRE11, GRE1, and GRE6 peptides. Quantitative immunofluorescence using a 96 well fluorometer demonstrated that the anti-GRE11 antibodies were inhibited or prevented from binding the GR epitopes by GRE11, partially with GRE1 and much less with GRE6. These observations were made on the basis of the following method. All samples were diluted in FTA buffer (PBS) including a negative Control, Rabbit Anti-diphtheria toxoid (DT) IgG (at 1:20); and an affinity purified Anti-GRE11.

The anti-GRE11 antibody and control (DT) antibodies were incubated in a mixture with 1: GRE11, 2: GRE1, 3: GRE1+6 (mix 1:1), 4: GRE6 (AA 1-12 of the gastrin receptor), 5: GnRH (negative control), for one hour at RT in a humid environment.

About 10 mg/ml Hoechst 33342 Trihydrochloride Trihydrate dye was diluted 1:1000 in FTA.

H69 cells were harvested from a t-flask and suspended with approximately 10 ml of Hoechst dye in a centrifugation tube. A 10 μl cell aliquot was counted with the hemocytometer. The remaining cells were stored for 30 minutes on ice.

The cells were washed once by centrifugation and resuspended in FTA to make 5×10⁶ cells/ml.

About 200 μl of cell suspension was tested at 1×10⁶ cells/tube with antibody plus peptide on ice for 45-50 minutes. After removal of supernatant and washing with FTA, 200 μl of FITC-F(ab)₂ of Goat α-Rabbit IgG diluted 1:50 in FTA, containing 10% Sigma Normal Goat Serum, was added to each tube.

The cells were resuspended in this secondary solution and incubated on ice, in the dark for 45-50 minutes.

The cells were washed with 200 μl of FTA buffer two times by centrifugation and aspiration of the wash buffer each time.

200 μl of FTA was added to each tube, and the cells were resuspended. The cells were the plated at aliquots of 100 μl in duplicate wells on a black Maxisorp plate and read using a fluorometer at the Hoechst wavelength setting, and also read at the FITC wavelength setting. The ratio of mean FITC/Hoechst fluorescence was calculated and the anti-DT value (the assay baseline) was subtracted from each. The percent inhibition of binding by each GRE peptide was calculated relative to binding in the presence of GnRH for the anti-GRE-11 antibody treated cells.

Results:

The percent inhibition of antibody binding by each peptide was: 84% GRE-11, 57% GRE1, 49% GRE1+GRE6 (mix) and 4% GRE6.

Thus, strong inhibition was obtained with GRE-11, with significant inhibition by GRE1; this shows that GRE11 possesses additional epitopes in common with the gastrin receptor, over those shared by GRE1 and GRE6 with the receptor. This suggests that GRE-11 sequence is a surprisingly effective peptide as an anti-gastrin receptor immunogen. The anti-GRE-11 Ab have been shown in vitro to inhibit the proliferation of GR+AR42J cells (rat, pancreatic cancer line) in comparison with anti-DT Ab, as follows:

The AR42J cells were harvested from sub-confluent T75 flasks using 0.025% EDTA and plated in 96 well plates (100 μl/well of 1×10⁵ cells/mL). The cell culture media were prepared from RPMI 1640 with 2 mM L-glutamine and 1% FBS. After 24 hours, affinity purified anti-GRE-11 Ab or protein A purified normal rabbit IgG were added to a concentration of 500 μg/mL in cell culture media. After 3 days of culture in the presence of the antibody, cell proliferation was assessed by the MTT assay.

It was found that the anti-GRE-11 antibody reduced the growth of AR42J cells by 25% in the three day assay.

In addition, the anti-GRE-11 Ab have also been found to induce apoptosis of the GR+ tumor cell line in vitro.

The AGS tumor line normally has a low expression level of GR; however, a variant of the line transfected with the human GR, expresses high levels of the GR. This line is designated AGSCCK-2R. The effects of affinity purified rabbit Anti-GRE11 Ab on apoptosis of AGS-CCK-2r were compared with protein A purified normal rabbit IgG as control. Vector control AGSvc cells were tested as additional controls. Apoptosis was measured by the Tunnel method as follows:

AGS cells (AGS-gr and AGSvc) were harvested from sub-confluent T75 flasks using 0.025% EDTA and plated out in 24 well plates at 1×10⁵ cells per well in RPMI 1640 media with 2 nM L-glutamine and 10% FBS. Each well contained a 13 mm diameter tissue culture treated coverslip. After 24 hours the media was replaced with RPMI plus 2 nM L-glutamine and 1% FBS and the test antibodies (GRE-11 or rabbit IgG control at 500 μg/mL). At 18 hours, the cells attached to the coverslips were fixed in situ in 4% formaldehyde (in PBS) for 10 minutes, prior to labeling with the TdT-FragEL® DNA fragmentation detection kit (Oncogene Research Products, QIA33). The kit allows for the detection of apoptotic nuclei by binding terminal deoxynucleotidyl transferase (TdT) to exposed 3′-OH ends of DNA fragments generated in response to apoptotic signals. TdT catalyses the addition of biotin labeled and unlabelled deoxynucleotides to the fragments , which is visualized by DAB chromogen via strepavidin horseradish peroxidase anti-biotin antibody conjugate.

The cells were rehydrated with TBS and permeabilized with 20 μg Proteinase K for 5 minutes and then endogenous peroxidases were inactivated with 3% hydrogen peroxide for 5 minutes. The cells were then treated with TdT enzyme for 90 minutes at 37° C. The reaction was halted with stop solution and incubated with blocking buffer prior to HRP conjugation. DAB was applied followed by methyl green counterstaining.

The coverslips were removed from the wells and mounted onto glass slides and coverslipped with glass using standard mounting media. Image analysis was conducted using Qwin Standard (Leica, Germany) and the number of apoptotic cells were assessed for each treatment. The results are given as mean percentage of apoptosis over 20 readings for each slide and 20× objective magnification. Basal rates of apoptosis were <1% in the AGSvc cells. Treatment with anti-GRE-11 antibodies caused a significant 2.4 fold (p<0.05) increase of apoptosis in AGS-gr cells compared to purified normal rabbit IgG.

The results were analyzed and showed that the mean percent apoptosis (±SE) for each group were AGSvc with normal rabbit IgG: 0.37%±0.07; AGSvc with anti-GRE-11: 0.83%±0.114; AGS-gr with normal rabbit IgG: 0.86%±0.14; and AGS-gr with anti-GRE-11: 2.07%±0.27. Thus, a significant increase in apoptosis by anti-GRE11 antibody was observed in the gastrin receptors transfected cells but not in the control cells.

Peptides GRE-9 and 10 are internal splice variants of the third internal domain of the mutant human CCK-2/gastrin receptor. Receptors detected by anti-GRE9 or anti-GRE10 antibodies may be unique to certain tumor cells.

### EXAMPLE 15

Monoclonal antibodies to the gastrin receptor were produced using immunogen of this invention.

The peptide comprising GRE11 (SEQ ID NO:11) was linked to DT as described in Example 13 to produce GRE11-DT conjugate. Immunogens were prepared with the GRE11-DT conjugate using Montanide ISA 703 as described for GRE1-DT in Example 1. Adult CAF1 strain mice were immunized with 0.1 mg of the GRE11-DT immunogen by injection of 0.1 mL/mouse, by the intraperitoneal (IP) route. The mice were given a second injection 4 weeks later.

Four days prior to cell fusion, the mice were boosted with 0.1 mg of conjugate in PBS by IP injection. On the day of fusion, the spleens were harvested and used as a source of antibody producing cells, which were fused with mouse P3 cells by standard hybridoma methods practiced by those skilled in the art.

Hybridomas producing monoclonal antibodies against the gastrin receptor were selected for based on antibody binding in two assays. In the first assay, cell culture supernatants were tested for the presence of antibody to the GRE11 peptide in and ELISA, which was conducted as that described in Example 1 for GRE1 antibodies, excepting that GRE11-BSA served as the antigenic target for the anti-GRE11 antibodies. By this method, cells producing antibodies to the GRE11 peptide were identified. For example, in fusion #446, there were 41 wells out of 576 wells found to contain hybrid cells producing anti-GRE11 peptide antibodies.

These cells were then subjected to the second selective step, wherein they were tested for production of antibody that bound to the gastrin receptor on receptor positive cells. An immunofluorescence assay (IFA) was used to identify anti-gastrin receptor antibodies.

The following method was used for the IFA. Gastrin positive cells are grown under tissue culture condintions recommended for the particular cell line by methods known to those skilled in the art. Examples of such cell lines would include, but not be limited to, H69, C170 HM2, AGS, AGS transfected with human gastrin receptor and NIH 3T3 cells transfected with human gastrin receptor, etc. On the day prior to the IFA, gastrin receptor positive cells were harvested (such as gastrin receptor transfected AGS cells) from one T150 flask. A single cell suspension was prepared and the cells were counted. The cell concentration was adjusted to ˜0.5-1 million cells per mL. Twelve (12) well culture slides were flooded with cell suspension in a sterile petri dish, then incubated at room temperature under sterile conditions for ˜1 hour. The slides were immersed in complete DMEM (Dulbecco's MEM), then incubated at 37° C., under 5% CO₂ overnight. The slides were removed from the petri dishs and rinsed in PBS for 1-2 minutes. Next, the slides were immersed in paraformaldehyde fixative for 60 minutes, then twice rinsed in PBS, 5 minutes per rinse. The excess PBS was removed by shaking and the slide flooded with 1% BSA in PBS, then incubated for 1 hour at room temperature in a moist slide chamber. The 1% BSA in PBS was decanted and 25 μl of controls (mouse anti-GRE11 antiserum and nonspecific negative mouse serum control) and test samples (supernatant samples rom hybrid cell wells) were added to individual wells. The slides were incubated for 60 minutes at room temp in a humid slide chamber. The slides were washed for 5 minutes by adding slides to a staining jar filled with PBS. This was repeated one more time. The slides were flooded with a 1:40 dilution of FITC sheep anti-mouse IgG, M & A (H+L) conjugate, and incubated at room temperature in the dark for 1 hour in a humidified slide chamber. The slides were washed for 5 minutes by adding the slides to a staining jar filled with PBS buffer; this was repeated once. The slides were flooded with mounting medium and a 20×60 mm cover slip was placed on each slide. Individual wells were viewed under a fluorescent microscope and the cells were visually assessed in each well on the slides for staining by the monoclonal antibodies.

For example, in fusion #446, only 4 hybrids were found to be producing antibodies that bound to gastrin receptor on cell membrane, out of the 41 hybrids making anti-GRE11 peptide antibody. Cells thus identified as producing anti-gastrin receptor antibodies were then cloned three times, per standard hybridoma techniques, to yield monoclonal hybridomas producing monoclonal anti-gastrin receptor antibodies. Following each cloning, the hybrid cells were re-tested as described above for anti-gastrin receptor monoclonal antibody production. It is noted that alternative appropriate methods known to those skilled in the art, such as radioimmunoassay, cell targeting ELISA, Western blot, etc., can alternatively be utilized to identify hybrid cells making specific, high affinity antibodies to the gastrin receptor.

For another example, by the techniques, as described, hybrid lines were produced from fusions #446 and #447 which secrete monoclonal anti-gastrin receptor antibodies, numbered accordingly 446-1, -2, -3, and 447-1, respectively.

This methodology can be further employed to select specific targets in the extracellular moiety of the GR.

### EXAMPLE 16

Combination treatment of immunization against GR and chemotherapy was tested in rats. Results are reported in Table 5.

Subject: Rats (BDIX Strain)

Method: Rats (BDIX strain) received 7 immunizations with GRE1 or control immunogens (injected on weeks −4, −3, −2, 0, 1, 4, 7).

All rats were injected with 10⁶ DHDK 12 rat tumor cells at wk 0.

Some groups were treated with 5FU/leucovorin at wks 1 and 5.

All rats were terminated week 10 and tumors were assessed.

TABLE 5

| | # | | | Prolif: | % CCK-2 |
|---|---|---|---|---|---|
| | rats w/ | # | Mean % | % BrdU | GRE1 |
| Group | tumors | tumors | Necrosis* | Staining | Staining |
| Control Imm | 3 | 4 | 19 ± 3 | 15 ± 1 | 48 ± 3 |
| GRE1 Imm | 7 | 15 | 28 ± 2 | 5 ± 0.1 | 24 ± 2 |
| 5FU/Leu | 8 | 21 | 35 ± 2 | 15 ± 0.4 | 55 ± 2 |
| 5Fu/Leu + GRE1 | 2 | 9 | 63 ± 2 | 1 ± 0.2 | 15 ± 1 |

*± s.e. of mean

We found a relatively low take rate in some groups. The differences were statistically significant (Mann Whitney) for each parameter measured.

Results Showed:

Immunization with GRE1 epitope increased tumor necrotic area and reduced both proliferation and gastrin receptor expression level of tumor cells in comparison with rats receiving control immunogen.

These effects were markedly enhanced by co-treatment with 5FU and leucovorin.

Conclusion:

Immunization with GRE1 was therapeutically effective. Combination of GRE1 immunization with chemotherapy significantly enhanced efficacy over either treatment alone.

### REFERENCES

1. Bock, M. G., DiPardio, R. M., Evans, B. E., Rittle, K. E., Whitter, A., Veber, D, Anderson, E., and Freidinger, A. Benzodiazepine, gastrin and brain cholecystokinin receptor ligands: L-365,260. J. Med. Chem., 32: 13-17, 1989.

2. Curtis, B. M., Widmer, M. B., de Roos, P., Qwarnstrom, E. E. IL-1 and its receptor are translocated to the nucleus. J. Immunol. 1990; 144:1295-1303.

3. Dickinson C. J. Relationship of gastrin processing to colon cancer (editorial). Gastroenterology 1995; 109:1384-1388.

4. Edkins, J. S. On the chemical mechanism of gastric secretion. Proc. Soc. Lond. 1905; 76:376.

5. Fourmy, D., Zahidi, A., Pradayrol, I., Vayssette, J., Ribet, A. Relationship of CCK/gastrin-receptor binding to amylase release in dog pancreatic acini. Regul. Pept. 1984; 10:57-68.

6. Grider, J. R., Malchlouf, G. M. Distinct receptors for cholecystokinin and gastrin on muscle cells of stomach and gallbladder. Am. J. Physiol. 1990; 259:G184-G190.

7. Harrison's Principles of Internal Medicine, Isselbacher et al. Eds. 13^{th} Ed. Pages 1690-1691, 1994.

8. Holt, S. J., Alexander, P., Iman, C., Davies, D. Epidermal growth factor induced tyrosine phosphorylating of nuclear proteins associated with translocation of epidermal growth factor receptor into the nucleus. Biochem. Pharm. 1994; 43:117-126.

9. Hughes, J., Boden, P., Costall, B., Domeney, A., Kelly, E., Horwell, D. C., Hunter, J. C., Pinnock, R. D., and Woodruff, G. N. Development of a class of selective cholecystokinin type B receptor antagonists having potent anxiolytic activity. Proc. Natl. Acad. Sci., 87: 6728-6732, 1990.

10. Johnson L. New aspects of the trophic actin of gastrointestinal hormones. Gastroenterology 1997; 72:788-792.

11. Kopin, A. S., Lee, Y. M., McBride, E. W., Miller, L. J., Lu, M., Lin, H. Y., Kolakowski, L. F., Beinborn, M. Expression cloning and characterization of the canine parietal cell gastrin-receptor. Proc. Nat. Acad. Sci. USA 1992; 89:3605-3609.

12. Laudron P. M. From receptor internalization to nuclear translocation: new targets for long-term pharmacology. Biochem. Pharm. 1994; 47:3-13.

13. Le Meuth, V., Philouz-Rome, V., LeHuerou-Luron, L., Formal, M., Vaysse, N., Gespach, C., Guilloteau, P., Fourmy, D. Differntial expression of A- and B-subtypes of cholecystokinin/gastrin-receptors in the developing calf pancreas. Endocrinology 1993; 133:1182-1191.

14. Matsumoto, M., Park, J., Yamada, T. Gastrin-receptor characterization: affinity cross-linking of the gastrin-receptor on canine gastric parietal cells. Am. J. Physiol. 1987; 252:G143-147.

15. Nakata, H., Matsui, T., Ito, M., Taniguchi, T., Naribayashi, Y., Arima, N., Nakamura, A., Kinoshita, Y., Chibara, K., Hosoda, S., Chiba, T. Cloning and characterization of gastrin-receptor from ECL carcinoid tumor of Mastomys natalensis. Biochem. Biophys. Res. Commun. 1992; 187:1151-1157.

16. Narayan, S., Chicone, L., Singh, P. Characterization of gastrin binding to colonic mucosal membranes of guinea pigs. Mol. Cell. Biochem. 1992; 112:163-171. Nemeth, J., Taylor, B., Pauweis, S., Varro, A & Dockray, G. J. Identification of progastrin derived peptides in colorectal carcinoma extracts. Gut 1993; 34: 90-95.

17. Palnaes, Hansen C., Stadil, F., Rehfeld, J. F. Metabolism and influence of glycine-extended gastrin on gastric acid secretion in man. Digestion 1996; 57:22-29.

18. Podlecki, D. A., Smith. R. M., Kao, M., Tsai, P., Huecksteadt, T., Brandenburg, D., Lasher, R. S., Jarett, L., Olefsky, J. M. Nuclear translocation of the insulin receptor. J. Biol. Chem. 1987; 262:3362-3368.

19. Rehfeld, J. F., Bardram, L., Hilsted, L. Gastrin in human bronchogenic carcinomas: constant expression but variable processing of progastrin. Cancer Res. 1989; 49:2840-2843. Rehfeld, J. F. Three components of gastrin in human serum. Biochim, Biophys. Acta 1972, 285: 364-372.

20. Romani, R., Howes, L. G., and Morris, D. L. Potent new family of gastrin-receptor antagonists (GRAs) produces in vitro and in vivo inhibition of human colorectal cancer cell lines. Procs. AACR, 35: 397 (Abstract), 1994.

21. Scemma, J. L., Fourmy, A., Zahidi, L., Praydayrol, L., Susini, C., Ribet, A. Characterization of gastrin-receptors on a rat pancreatic acinar cell line (AR4-2J). A possible model for studying gastrin mediated cell growth and proliferation. Gut 1987; 28:233-236.

22. Seva, C., Dickinson, C. J., Yamada, T. Growth-promoting effects of glycine-extended prograstrin. Science 1994; 265:410-412. Seva, C., Dickinson, C. J., Sawada, M., Yamada, T. Characterization of the glycine-extended gastrin (G-gly) receptor on AR 42Z cells. Gastroenterology 1995, A742.

23. Singh, P., Owlia, A., Espeijo, R., Dai, B. Novel gastrin-receptors mediate mitogenic effects of gastrin and processing intermediates of gastrin on Swiss 3T3 fibroblasts. Absence of detectable cholecystokinin (CCK)-A and CCK-B receptors. J. Biol. Chem. 1995; 270:8429-8438.

24. Singh, P., Townsend, Jr. C. M., Thompson, J. C., Narayan, S., Guo, Y. S. Hormones in colon cancer: past and prospective studies. Cancer J. 1990; 3:28-33.

25. Soll, A. H., Amiran, L. P., Thomas, T., Reedy, T. J., Elashoff, J. D. Gastrin-receptors on isolated canine parietal cells. J. Clin. Invet. 1984; 73:1434-1447.

26. Taniguchi, T., Matsui, T., Ito, M., Marayama, T., Tsukamota, T., Katakami, Y., Chiba, T., Chihara, K. Cholecystokinin-B/gastrin-receptor signaling pathway involve styrosine phosphorylatins of p125FAK and p42MAP. Oncogene 1994; 9:861-867.

27. Todisco, A., Takeuchi, Y., Seva, C., Dickinson, C. J., Yamada, T. Gastrin and glycine-extended progastrin processing intermediates induce different programs of carly gene activation. J. Biol. Chem. 1995; 279:28337-28341.

28. Ulrich, A., Schlessinger, J. Signal transduction by receptors with tyrosine kinase activity. Cell 1990; 61:203-212. Upp, J. R., Singh, S., Townsend, C. M., and Thompson, J. C. Clinical significance of gastrin receptors in human colon cancers. Cancer Res. 1989, 49: 488-492.

29. Van-Solinge, W. W., Nielsen, F. C., Friis-Hansen, L., Falkmer, U. G. and Rehfeld, J. F. Expression but incomplete maturation of progastrin in colorectal carcinoma. Gastroenterology 1993; 104:1099-1107.

30. Wank, S. A. Cholecystokinin receptors (editorial). Am. J. Physiol. 1995; 269:G628-G646.

31. Wank, S. A., Pisegna, J. R., de Weerth, A. Brain and gastrointestinal cholecystokinin receptor family: structure and functional expression. Proc. Nat. Acad. Sci. USA 1992; 89:8691-8695.

32. Watson, S. A., Durrant, L. G., Crosbie, J. D, Morris D. L. The in vitro growth response of primary human colorectal and gastric cancer cells to gastrin. Int. J. Cancer 1989; 43:692-696.

33. Watson, S. A., Durrant, L. G., Elston, P., and Morris, D. L. Inhibitory effects of the gastrin-receptor antagonist (L-365,260) on gastrointestinal tumor cells. Cancer, 1991, 68,1255-1260.

34. Watson, S. A., Crosbie, D. M., Moris, D. L., Robertson, J. F., Makovec, F., Rovati, L. C. et al. Therapeutic effect of the gastrin receptor antagonist, CR2093 on gastrointestinal tumour cell growth. B. J. Cancer 1992, 65(6): 879-883.

35. Watson, S. A., Morris, D. L., Durrant, L. G., Robertson, J. F., Hardcastle, J. D. Inhibition of gastrin-stimulated growth of gastrointestinal tumour cells by octreatide and the gastrin/cholecystokinin receptor antagonists, proglumide and lorglumide. Eur. J. Cancer 1992, 28A (8-9): 1462-1467.

36. Watson, S. A., Steele, R. Gastrin-receptors on gastrointestinal tumor cells, In: Gastrin-receptors in gastrointestinal Tumors. R. G. Landes Co., Austin TX:CRC 1993 p. 20-

37. Waston, S. A., Michaeli, D., Grimes, S., Morris, T. M., Robinson, G., Varro, A., Justin, T. A., Hardcastle, J. D., Gastrinmune raises antibodies that neutralise amidated and glycine-extended gastrin-17 and inhibit the growth of colon cancer. Cancer Res. 1996, 56: 880-885.

We claim:

1. An immunogen comprising:
a gastrin receptor-peptide epitope (GRE) selected from the group consisting of the synthetic sequences,
KLNRSVQGTGPGPGASLAAC (SEQ ID NO: 2),
CCGKLNRSVQGTGPGPGASL (SEQ ID NO: 5),
MELLKLNRSVQGC (SEQ ID NO: 8),
RDBDLGEADVWRASSC (SEQ ID No: 9),
WERRSGGNWAGDWGDSPFSSC (SEQ ID No: 10),
MELLKLNRSVQGTGPGPGASLC (SEQ ID No: 11),
MELLKLNRSVQGTGPGPGASLSSPPPPC (SEQ ID NO: 12),
ELLKLNRSVQGTGPGPGASLC (SEQ ID NO: 13),
LLKLNRSVQGTGPGPGASLC (SEQ ID NO: 14),
LKLNRSVQGTGPGPGASLC (SEQ ID NO: 15),
KLNRSVQGTGPGPGASLC (SEQ ID NO: 16),
ELLKLNRSVQGSSC (SEQ ID NO: 17),
and GTGPGPGASLC (SEQ ID NO: 18),
conjugated at its cysteine end to an immunogenic carrier.

2. An immunogen comprising:
GRE selected from the group consisting of SEQ ID NO: 5, 11, 12, 13, 14, 15, 16, 17, and 18, conjugated to an immunogenic carrier.

3. An immunogen comprising:
a GRE consisting of MELLKLNRSVQGC (SEQ ID NO: 8), conjugated to an immunogenic carrier.

4. An immunogen comprising:
a GRE consisting of amino acid sequence SEQ ID NO: 9 or 10, conjugated to an immunogenic carrier.

5. An immunogenic composition comprising the immunogen according to anyone of the claims 1-4, and a pharmaceutically acceptable carrier or adjuvant.

6. An antibody capable of binding the gastrin receptor immunomimic peptide selected from the group consisting of a sequences identified by SEQ ID NO: 2, 5, 6, 9, 10, 11, 12, 13, 14, 15, 16, 17 and 18.

7. The antibody according to claim 6 which is monoclonal antibody.

8. The antibody according to claim 7 which is murine, humanized or human.

9. A composition comprising one or more than one of the antibody of claim 7 or 8.

10. A composition for preventing or treating gastrin stimulated malignant or premalignant growth comprising an antibody prepared from an immune serum or supernatant which is specific for a gastrin receptor epitope consisting of an amino acid sequence identified as SEQ ID NO: 2, 5, 8-17 or 18.

11. A composition for preventing or treating gastrin stimulated malignant or premalignant growth comprising an antibody prepared from an immune serum or supernatant which is specific for the tumor gastrin receptor epitope consisting of an amino-acid sequence listed as SEQ ID NO: 9 or 10.

12. The composition of the claims 10 or 11, wherein the antibody is conjugated to a cytotoxic substance.

13. The composition of the claim 12, wherein the cytotoxic substance comprises a toxin or radioactive substance.

14. The composition of the claim 13, wherein the toxin is a cholera toxin, diphtheria toxin, or ricin; and the radioactive substance is ¹²⁵Iodine, ¹³¹Iodine, ⁹⁹Yttrium or ¹¹¹Indium.

15. A method for the diagnosis of the gastrin receptor in a biopsy comprising the steps of:
(i) obtaining a biopsy specimen from a patient,
(ii) exposing the specimen to an anti-GR antibody prepared from an immune serum or supernatant, the antibody being specific for a gastrin receptor peptide epitope consisting of an amino acid sequence listed as SEQ ID NO: 2, 5, 8-17 or 18; and
(iii) detecting the bound antibody by a colorimetric, chemilumenescent, fluorescent, radiometric or scintigraphic technique.

16. The method for detection of gastrin responsive malignant or premalignant tumor in the patient, comprising: administration of anti-GR antibodies conjugated to a detectable molecule comprising a colorimetric, chemilumenescent, or radioactive molecule, and imaging of the antibody complexes by imaging techniques.

17. A method of treatment of a patient suffering from a gastrin responsive tumor, comprising:
(i) administering a therapeutically effective amount of animal, human or humanized anti-GRE 11 antibodies, which may be modified to carry a chemotherapeutic agent or a radioactive substance, and
(ii) administering a therapeutically effective amount of gastrin G17 immunogen and/or
(iii) a therapeutically effective amount of animal, human or humanized anti-G17 antibodies.

18. The method of claim 17 wherein the antibodies are a single monoclonal species or a mixture of different monoclonal species.

19. A liposomal composition comprising a liposomal vesicle suspension containing the antibody as claimed in claim 6.

20. A method of treatment against cancer comprising a combination of administering:
(i) an immunogen against a gastrin receptor epitope as claimed in claim 1 or 2; and/or
(ii) an antibody as claimed in claim 6; and
(iii) a chemotherapeutic agent selected from the group consisting of 5FU (+leucovorin), gemcitabine, irinatecan, taxane, oxiplatin, carboplatin, cisplatin, camptothecin/camptosar, vincristin, vinblastine, rubetecan, cyclophosphamide, doxirubicin, mitomycin C, etoposide and noscapine.


SEQUENCE LISTING (18 sequences)

SEQ ID NO: 1 - protein, 17 aa, Homo sapiens
  PEPTIDE (1)..(17): Amino acid residues 5-21 of the CCK-B/Gastrin receptor
KLNRSVQGTGPGPGASL

SEQ ID NO: 2 - protein, 20 aa, Homo sapiens
KLNRSVQGTAPGPGASLAAC

SEQ ID NO: 3 - protein, 8 aa, Homo sapiens
  PEPTIDE (1)..(7): Synthetic peptide spacer
SSPPPPPC

SEQ ID NO: 4 - protein, 24 aa, Homo sapiens
  PEPTIDE (1)..(17): Amino acid residue 5-21 of the CCK-B/Gastrin receptor
KLNRSVQGTGPGPGASLSSPPPPC

SEQ ID NO: 5 - protein, 20 aa, Homo sapiens
  PEPTIDE (4)..(20): Amino acid residues 5-21 of the CCK-B/Gastrin receptor
CGGKLNRSVQGTGPGPGASL

SEQ ID NO: 6 - protein, 15 aa, Homo sapiens
GPGAHRALSGAPISF

SEQ ID NO: 7 - protein, 22 aa, Homo sapiens
  PEPTIDE (16)..(22): Synthetic peptide spacer
GPGAHRALSGAPISFSSPPPPC

SEQ ID NO: 8 - protein, 13 aa, Homo sapiens
MELLKLNRSVQGC

SEQ ID NO: 9 - protein, 16 aa, Homo sapiens
RDQDLGEADVWRASSC

SEQ ID NO: 10 - protein, 21 aa, Homo sapiens
WERRSGGNWAGDWGDSPFSSC

SEQ ID NO: 11 - protein, 22 aa, Homo sapiens
MELLKLNRSVQGTGPGPGASLC

SEQ ID NO: 12 - protein, 28 aa, Homo sapiens
  PEPTIDE (22)..(28): Synthetic spacer peptide
MELLKLNRSVQGTGPGPGASLSSPPPPC

SEQ ID NO: 13 - protein, 21 aa, Homo sapiens
ELLKLNRSVQGTAPGPGASLC

SEQ ID NO: 14 - protein, 20 aa, Homo sapiens
LLKLNRSVQGTGPGPGASLC

SEQ ID NO: 15 - protein, 19 aa, Homo sapiens
LKLNRSVQGTGPGPGASLC

SEQ ID NO: 16 - protein, 18 aa, Homo sapiens
KLNRSVQGTGPGPGASLC

SEQ ID NO: 17 - protein, 14 aa, Homo sapiens
ELLKLNRSVQGSSC

SEQ ID NO: 18 - protein, 11 aa, Homo sapiens
GTGPGPGASLC